(19) United States
(12) Patent Application Publication
(10) Pub. No.: US 2024/0175951 A1
(43) Pub. Date: May 30, 2024

(54) SYSTEMS AND METHODS FOR IMAGING AND TREATMENT

(71) Applicant: SHANGHAI UNITED IMAGING HEALTHCARE CO., LTD., Shanghai (CN)

(72) Inventors: Jianfeng LIU, Shanghai (CN); Xing'en YU, Shanghai (CN); Xiaofei HUANG, Shanghai (CN); Jian GU, Shanghai (CN); Cheng NI, Shanghai (CN)

(73) Assignee: SHANGHAI UNITED IMAGING HEALTHCARE CO., LTD., Shanghai (CN)

(21) Appl. No.: 18/398,293

(22) Filed: Dec. 28, 2023

Related U.S. Application Data
(63) Continuation of application No. PCT/CN2021/137181, filed on Dec. 10, 2021.

(51) Int. Cl. G01R 33/38; A61N 5/10; G01R 33/36

(52) U.S. Cl. CPC G01R 33/3804; G01R 33/3642; A61N 5/10

(57) ABSTRACT

The present disclosure may provide a system. The system may include a magnetic resonance imaging (MRI) device configured to perform an imaging of a subject. The MRI device may includes a cryostat. The cryostat may include a first cooling chamber and a second cooling chamber that are in fluid communication through a connection conduit. The connection conduit may be located on a side of a central axis of the first cooling chamber or a central axis of the second cooling chamber. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam.

### CROSS-REFERENCE TO RELATED APPLICATIONS

This application is a Continuation of International Application No. PCT/CN2021/137181, filed on Dec. 10, 2021, the contents of which are hereby incorporated by reference.

### TECHNICAL FIELD

The present disclosure generally relates to medical technology, and more particularly, systems and methods for imaging and treatment.

### BACKGROUND

An imaging device (e.g., a magnetic resonance imaging (MRI) device) and a radiotherapy device (e.g., a linear accelerator) are combined to perform an image-guided radiation therapy (IGRT) of a subject. In some cases, a radiation beam emitted by the radiotherapy device may impinge on the subject after having traversed certain parts (e.g., a superconducting magnet, a cooling medium) of the MRI device, which may cause an attenuation of the radiation beam, and an amount of the radiation beam impinging on the subject may be uncontrollable, thereby resulting in an inaccurate delivery of radiation treatment. Besides, the superconducting magnet of the MRI device may lose the superconductivity due to the radiation of the radiation beam. Thus, it is desirable to develop systems and methods for imaging and treatment with improved efficiency and reliability.

### SUMMARY

According to one aspect of the present disclosure, a system may be provided. The system may include a magnetic resonance imaging (MRI) device configured to perform an imaging of a subject. The MRI device may includes a cryostat. The cryostat may include a first cooling chamber and a second cooling chamber that are in fluid communication through a connection conduit. The connection conduit may be located on a side of a central axis of the first cooling chamber or a central axis of the second cooling chamber. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam.

In some embodiments, the connection conduit may be filled with a liquid-state cooling medium filled in a portion of the first cooling chamber and a portion of the second cooling chamber.

In some embodiments, the connection conduit may be located below the central axis of the first cooling chamber or the central axis of the second cooling chamber.

In some embodiments, the first cooling chamber may have a first annular structure. A first end of the connection conduit may be located at a position within a first arc of the first annular structure. The second cooling chamber may have a second annular structure. A second end of the connection conduit may be located at a position within a second arc of the second annular structure.

In some embodiments, at least one superconducting wire may be housed inside the connection conduit. At least one first coil may be housed inside the first cooling chamber. At least one second coil may be housed inside the second cooling chamber. The at least one superconducting wire may be configured to operably connect the at least one first coil and the at least one second coil.

In some embodiments, the system may include a radiation protection component housed inside the connection conduit and configured to protect the at least one superconducting wire from being exposed to the radiation beam.

In some embodiments, the radiation protection component may include a pipe.

In some embodiments, the connection conduit may include a pipe.

In some embodiments, the first cooling chamber and the second cooling chamber may be in gas communication through a second connection conduit.

In some embodiments, the second connection conduit may be located outside the first cooling chamber and the second cooling chamber.

In some embodiments, at least a portion of the second connection conduit may be located above the radiation source.

In some embodiments, a first end of the second connection conduit may be operably connected to an upper portion of the first cooling chamber. A second end of the second connection conduit may be operably connected to an upper portion of the second cooling chamber.

In some embodiments, a first end of the second connection conduit may be operably connected to the first cooling chamber on a far side of the first cooling chamber that is farther away from the second cooling chamber than a near side of the first cooling chamber. A second end of the second connection conduit may be operably connected to an upper portion of the second cooling chamber.

In some embodiments, the system may include a vacuum layer housed outside the second connection conduit.

In some embodiments, a first end of the second connection conduit may be located inside a portion of the first cooling chamber filled with a gaseous-state cooling medium. A second end of the second connection conduit may be located inside a portion of the second cooling chamber filled with the gaseous-state cooling medium.

In some embodiments, a length direction of the connection conduit may be parallel with a length direction of the second connection conduit.

In some embodiments, the second connection conduit may be located inside the connection conduit.

In some embodiments, the connection conduit may be independent from the second connection conduit.

In some embodiments, the connection conduit may be made of metal.

In some embodiments, the second connection conduit may be made of stainless steel or a radiation impermeable material.

In some embodiments, the first cooling chamber may include a first quenching valve. The second cooling chamber may include a second quenching valve different from the first quenching valve.

In some embodiments, the first cooling chamber and the second cooling chamber may share a cold head.

In some embodiments, the first cooling chamber may include a first cold head. The second cooling chamber may include a second cold head different from the first cold head.

In some embodiments, at least a portion of the first cooling chamber may be filled with a cooling medium; or at least a portion of the second cooling chamber may be filled with the cooling medium.

In some embodiments, the cooling medium includes liquid helium.

According to another aspect of the present disclosure, a system may be provided. The system may include a magnetic resonance imaging (MRI) device configured to perform an imaging of a subject. The MRI device may include a cryostat. The cryostat may include a first cooling chamber and a second cooling chamber. The first cooling chamber and the second cooling chamber may be in gas communication through a connection conduit. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam. The connection conduit may be positioned out of a rotation range of the radiation source.

In some embodiments, at least a portion of the connection conduit may be located above the radiation source.

In some embodiments, a first end of the connection conduit may be operably connected to an upper portion of the first cooling chamber. A second end of the connection conduit may be operably connected to an upper portion of the second cooling chamber.

In some embodiments, a first end of the connection conduit may be operably connected to the first cooling chamber on a far side of the first cooling chamber that is farther away from the second cooling chamber than a near side of the first cooling chamber. A second end of the connection conduit may be operably connected to an upper portion of the second cooling chamber.

In some embodiments, the system may include a vacuum layer housed outside the connection conduit.

According to another aspect of the present disclosure, a system may be provided. The system may include a magnetic resonance imaging (MRI) device configured to perform an imaging of a subject. The MRI device may include a cryostat. The cryostat may include a first cooling chamber and a second cooling chamber. The first cooling chamber and the second cooling chamber may be in gas communication through a connection conduit. A first end of the connection conduit may be located inside a portion of the first cooling chamber filled with a gaseous-state cooling medium. A second end of the connection conduit may be located inside a portion of the second cooling chamber filled with the gaseous-state cooling medium. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam.

According to another aspect of the present disclosure, a system may be provided. The system may include a magnetic resonance imaging (MRI) device configured to perform an imaging of a subject. The MRI device may include a cryostat. The cryostat may include a first cooling chamber and a second cooling chamber. The first cooling chamber may include a first quenching valve. The second cooling chamber may include a second quenching valve different from the first quenching valve. The first cooling chamber and the second cooling chamber may share a cold head. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam.

According to another aspect of the present disclosure, a system may be provided. The system may include a magnetic resonance imaging (MRI) device configured to perform an imaging of a subject. The MRI device may include a cryostat. The cryostat may include a first cooling chamber and a second cooling chamber. The first cooling chamber may include a first quenching valve. The second cooling chamber may include a second quenching valve different from the first quenching valve. The first cooling chamber may include a first cold head. The second cooling chamber may include a second cold head different from the first cold head. The system may include a radiation source configured to emit a radiation beam toward the subject. The radiation source may be positioned between the first cooling chamber and the second cooling chamber such that the first cooling chamber and the second cooling chamber are outside of a radiation range of the radiation beam.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating an exemplary MRI device according to some embodiments of the present disclosure;

FIG. 3A provides a section view illustrating an exemplary medical device according to some embodiments of the present disclosure;

FIG. 3B and FIG. 3C provide side views illustrating an exemplary medical device according to some embodiments of the present disclosure;

FIG. 3D provides section views illustrating portions of an exemplary medical device according to some embodiments of the present disclosure;

FIG. 3E provides an exemplary angle range according to some embodiments of the present disclosure;

FIG. 4 provides a section view illustrating an exemplary medical device according to some embodiments of the present disclosure;

FIG. 5 provides a section view illustrating an exemplary medical device according to some embodiments of the present disclosure;

FIG. 6 provides a section view illustrating an exemplary medical device according to some embodiments of the present disclosure;

FIG. 7A and FIG. 7B provide section views illustrating an exemplary medical device according to some embodiments of the present disclosure;

FIGS. 8A and 8B provide section views illustrating an exemplary medical device according to some embodiments of the present disclosure;

FIG. 9 provides a section view illustrating an exemplary medical device according to some embodiments of the present disclosure; and

FIG. 10 provides a section view illustrating an exemplary medical device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that the terms “system,” “device,” “assembly,” “component,” etc., when used in this disclosure, refer to one or more parts with one or more specific purposes. However, a structure that may perform a same or similar function compared to a part exemplified above or referred to elsewhere in the present disclosure may be named differently from the present disclosure.

In the present disclosure, spatial reference terms such as “center,” “longitudinal,” “transverse,” “length,” “width,” “thickness,” “upper,” “lower,” “front,” “back,” “left,” “right,” “vertical,” “horizontal,” “top,” “bottom,” “inner,” “outer,” “clockwise,” “counterclockwise,” “axial,” “radial,” “circumferential,” etc., indicate, in a relative sense, an orientation or positional relationship between two or more elements, assemblies, devices, or systems based on an orientation or positional relationship as shown in the drawings, and are only for the convenience and simplicity of description, rather than indicating or implying that the elements, assemblies, devices or systems in the present disclosure have a particular orientation when the disclosed system, or a portion thereof, is in operation, or are constructed and operated in a particular orientation, and therefore may be not understood as a limitation of the present disclosure.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

In the present disclosure, unless otherwise clearly specified and limited, the terms “mount,” “connect,” “couple,” “fix,” “locate,” “dispose,” etc., should be understood in a broad sense, for example, it may be a fixed connection, a detachable connection, integrated into a whole, a mechanical connection, an electrical connection, directly connected, or indirectly connected via an intermediate medium, an internal connection of two elements, or an interconnection of two elements, unless otherwise clearly defined. For those skilled in the art, the specific meanings of the above terms in the present disclosure may be understood according to specific circumstances.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

According to some embodiments of the present disclosure, a system may be provided. The system may include a radiation source and a magnetic resonance imaging (MRI) device. The MRI device may be configured to perform an imaging of a subject. The radiation source may be configured to emit a radiation beam toward the subject, for example, based on the imaging result of the subject to perform a radiation treatment. In some embodiments, the MRI device may include a cryostat. The cryostat may include a first cooling chamber and a second cooling chamber that is spaced apart from the first cooling chamber. The cryostat may also include a connection conduit that is configured to facilitate fluid communication between the first cooling chamber and the second cooling chamber. The first cooling chamber and/or the second cooling chamber may be at least partially filled with a cooling medium.

In existing application scenarios, a volume or a depth of a liquid-state cooling medium in a cooling chamber may change due to a change in a temperature of the liquid-state cooling medium. Besides, different volumes or depths of the liquid-state cooling medium through which a radiation beam passes may lead to different amounts of attenuation of the radiation beam, thereby causing the amount of the radiation beam impinging on a subject to be uncontrollable, which in turn may result in an inaccurate delivery of radiation treatment to the subject. In order to avoid or alleviate such problems, the radiation source in some embodiments of the present disclosure may be positioned between the first cooling chamber and the second cooling chamber, and the first cooling chamber and the second cooling chamber may be arranged outside a radiation range of the radiation beam, such that the radiation beam does not traverse the first cooling chamber and the second cooling chamber, and therefore not affected by a change in the volume or the depth of the liquid-state cooling medium inside the first cooling chamber and/or the second cooling chamber.

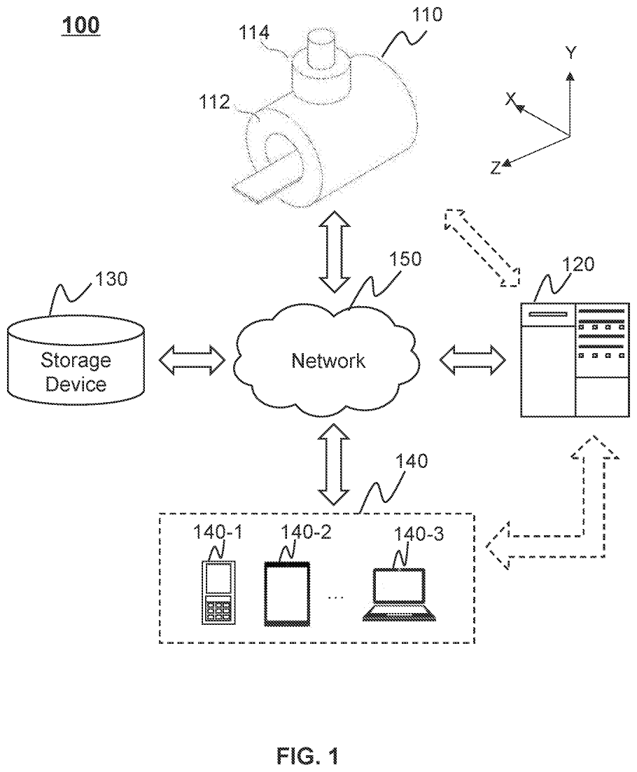

FIG. 1 is a schematic diagram illustrating an exemplary medical system according to some embodiments of the present disclosure. Merely by way of example, as illustrated in FIG. 1, the medical system 100 may include a medical device 110, a processing device 120, a storage device 130, one or more terminals 140, and a network 150. In some embodiments, the components in the medical system 100 may be connected to and/or communicate with each other in one or more of various ways. Merely by way of example, the medical device 110 may be connected to the processing device 120 through the network 150. As another example, the medical device 110 may be connected to the processing device 120 directly as indicated by the bi-directional arrow in dotted lines linking the medical device 110 and the processing device 120. As a further example, the storage device 130 may be connected to the processing device 120 directly or through the network 150. As still a further example, the terminal(s) 140 may be connected to the processing device 120 directly (as indicated by the bi-directional arrow in dotted lines linking the terminal(s) 140 and the processing device 120) or through the network 150.

In some embodiments, the medical device 110 may include a single-modality device, for example, an imaging device (e.g., a magnetic resonance imaging (MRI) device), a radiotherapy device (e.g., a linear accelerator), etc. In some embodiments, the medical device 110 may include a multi-modality device. In some embodiments, the multi-modality device may be configured to acquire image data relating to at least one part of a subject and perform treatment on the at least one part of the subject, etc. For example, the multi-modality device may include a first device 112 configured to generate an image including a representation of at least one part of a subject and a second device 114 configured to perform a treatment on at least one part of the subject. The first device 112 may include an MRI device (also referred to as a (magnetic resonance) MR device, an MR scanner), a magnetic resonance spectroscopy (MRS) device, etc. The second device 114 may include a radiotherapy device. The radiotherapy device may include a radiation source that is configured to generate and emit radiation beams to irradiate the subject in the treatment. Exemplary radiotherapy devices may include a linear accelerator, etc.

In some embodiments, the multi-modality device may be configured to acquire image data of different modalities. For example, the multi-modality device may include a first device and a second device each of which is configured to provide image data including a representation of at least one part of a subject. In some embodiments, the first device may be configured to generate a magnetic field in the acquisition of first image data. For example, the first device may include an MRI device (also referred to as an MR device, an MR scanner), a magnetic resonance spectroscopy (MRS) device, etc. The second device may include an imaging radiation source that is configured to generate and emit radiation beams to irradiate the subject in the acquisition of second image data. For example, the second device may include an X-ray imaging device, a computed tomography (CT) scanner, a digital radiography (DR) scanner (e.g., a mobile digital radiography), a digital breast tomosynthesis (DBT) scanner, a digital subtraction angiography (DSA) scanner, a dynamic spatial reconstruction (DSR) scanner, an X-ray microscopy scanner.

In some embodiments, the image data relating to at least one part of a subject may include an image (e.g., an image slice), projection data, or a combination thereof. For example, the image data of the subject may include a scout image associated with a body part of the subject. In some embodiments, the image data may be a two-dimensional (2D) imaging data, a three-dimensional (3D) imaging data, a four-dimensional (4D) imaging data, or the like, or any combination thereof. The subject may be biological or non-biological. For example, the subject may include a patient, a man-made subject, etc. As another example, the subject may include a specific portion, organ, tissue, and/or a physical point of the patient. Merely by way of example, the subject may include head, brain, neck, body, shoulder, arm, thorax, cardiac, stomach, blood vessel, soft tissue, knee, feet, or the like, or a combination thereof.

In the present disclosure, the X-axis, the Y-axis, and the Z-axis shown in FIG. 1 may form an orthogonal coordinate system. The X-axis and the Z-axis shown in FIG. 1 may be horizontal, and the Y-axis may be vertical. As illustrated, the positive X-direction along the X-axis may be from the right side to the left side of the medical device 110 seen from the direction facing the front of the medical device 110; the positive Y-direction along the Y-axis shown in FIG. 1 may be from the lower part to the upper part of the medical device 110; the positive Z-direction along the Z-axis shown in FIG. 1 may refer to a direction in which the subject is moved out of a detection region (or referred to as the bore) of the medical device 110.

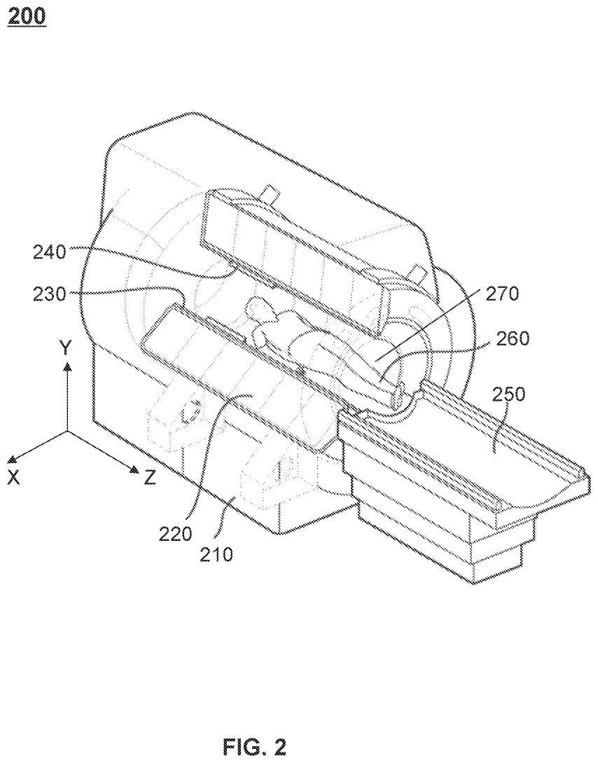

The following descriptions are provided regarding a multi-modality device including an MRI device and a radiotherapy device as the medical device 110 unless otherwise stated. It should be noted that the descriptions of the MRI device and the radiotherapy device in the present disclosure are merely provided for illustration, and not intended to limit the scope of the present disclosure. More descriptions for the multi-modality device may be found elsewhere in the present disclosure, for example, FIGS. 2-10 and the descriptions thereof.

The processing device 120 may process data and/or information obtained from the medical device 110, the storage device 130, and/or the terminal(s) 140. For example, the processing device 120 may be configured to obtain image data collected by the medical device 110 (e.g., an imaging device of the medical device 110). As another example, the processing device 120 may be configured to control the medical device 110 (e.g., a radiotherapy device of the medical device 110) to perform radiotherapy treatment based on the image data.

In some embodiments, the processing device 120 may be a computer, a user console, a single server, or a server group, etc. The server group may be centralized or distributed. In some embodiments, the processing device 120 may be local or remote. For example, the processing device 120 may access information and/or data stored in the medical device 110, the storage device 130, and/or the terminal(s) 140 via the network 150. As another example, the processing device 120 may be directly connected to the medical device 110, the storage device 130, and/or the terminal(s) 140 to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The storage device 130 may store data, instructions, and/or any other information. In some embodiments, the storage device 130 may store data obtained from the processing device 120 and/or the terminal(s) 140. For example, the storage device 130 may store image data (e.g., an MR image) collected by the medical device 110. As another example, the storage device 130 may store one or more algorithms for processing the image data, e.g., a magnetic resonance image reconstruction algorithm for MR image reconstruction, etc. In some embodiments, the storage device 130 may store data and/or instructions that the processing device 120 may execute or use to perform exemplary methods/systems described in the present disclosure. In some embodiments, the storage device 130 may include a mass storage, a removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random access memory (RAM). Exemplary RAM may include a dynamic RAM (DRAM), a double date rate synchronous dynamic RAM (DDR SDRAM), a static RAM (SRAM), a thyristor RAM (T-RAM), a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the medical system 100 (e.g., the processing device 120, the terminal(s) 140). One or more components in the medical system 100 may access the data or instructions stored in the storage device 130 via the network 150. In some embodiments, the storage device 130 may be directly connected to or communicate with one or more other components in the medical system 100 (e.g., the processing device 120, the terminal(s) 140). In some embodiments, the storage device 130 may be part of the processing device 120 or the medical device 110.

In some embodiments, a user and/or an operator may operate the medical system 100 using the terminal(s) 140. The terminal(s) 140 may include a mobile device 140-1, a tablet computer 140-2, a laptop computer 140-3, or the like, or any combination thereof. In some embodiments, the mobile device 140-1 may include a smart home device, a wearable device, a mobile device, a virtual reality device, an augmented reality device, or the like, or any combination thereof. In some embodiments, the smart home device may include a smart lighting device, a control device of an intelligent electrical device, a smart monitoring device, a smart television, a smart video camera, an interphone, or the like, or any combination thereof. In some embodiments, the wearable device may include a bracelet, a footgear, eyeglasses, a helmet, a watch, clothing, a backpack, a smart accessory, or the like, or any combination thereof. In some embodiments, the mobile device may include a mobile phone, a personal digital assistant (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™, etc. In some embodiments, the terminal(s) 140 may be part of the processing device 120.

The network 150 may include any suitable network that can facilitate the exchange of information and/or data for the medical system 100. In some embodiments, one or more components of the medical device 110, the processing device 120, the storage device 130, the terminal(s) 140, etc., may communicate information and/or data with one or more other components of the medical system 100 via the network 150. For example, the processing device 120 may obtain data from the medical device 110 via the network 150. As another example, the processing device 120 may obtain user instructions from the terminal(s) 140 via the network 150. The network 150 may be and/or include a public network (e.g., the Internet), a private network (e.g., a local area network (LAN), a wide area network (WAN))), a wired network (e.g., an Ethernet network), a wireless network (e.g., an 802.11 network, a Wi-Fi network), a cellular network (e.g., a long term evolution (LTE) network), a frame relay network, a virtual private network (VPN), a satellite network, a telephone network, routers, hubs, switches, server computers, and/or any combination thereof. Merely by way of example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the medical system 100 may be connected to the network 150 to exchange data and/or information.

It should be noted that the above description of the medical system 100 is merely provided for the purposes of illustration, and not intended to limit the scope of the present disclosure. For persons having ordinary skills in the art, multiple variations and modifications may be made under the teachings of the present disclosure. For example, the assembly and/or function of the medical system 100 may be varied or changed according to specific implementation scenarios.

FIG. 2 is a schematic diagram illustrating an exemplary MRI device according to some embodiments of the present disclosure. As illustrated, the MRI device 200 may include a gantry 210, a main magnet 220, gradient coils 230, radio frequency (RF) coils 240, and a patient support 250 (e.g., along the Z-direction). In some embodiments, the MRI device in the medical system 100 may be implemented based on the MRI device 200.

As illustrated, the main magnet 220 may generate a first magnetic field (or referred to as a main magnetic field) that may be applied to the subject 260 positioned inside the first magnetic field. The main magnet 220 may include a resistive magnet or a superconducting magnet that both need a power supply (not shown in FIG. 2) for operation. Alternatively, the main magnet 220 may include a permanent magnet. The main magnet 220 may form a detection region 270 and surround, along the Z-direction, the subject 260 that is moved into or positioned within the detection region 270. The main magnet 220 may also control the homogeneity of the generated main magnetic field. Some shim coils (not shown) may be in the main magnet 220. The shim coils placed in the gap of the main magnet 220 may compensate for the inhomogeneity of the magnetic field of the main magnet 220. The shim coils may be energized by a shim power supply.

The gradient coils 230 may be located inside the main magnet 220. For example, the gradient coils 230 may be located in the detection region 270. The gradient coils 230 may surround, along the Z-direction, the subject 260 that is moved into or positioned within the detection region 270. The gradient coils 230 may be surrounded by the main magnet 220 around the Z-direction, and be closer to the subject 260 than the main magnet 220. The gradient coils 230 may generate a second magnetic field (or referred to as a gradient field, including gradient fields Gx, Gy, and Gz). The second magnetic field may be superimposed on the main magnetic field generated by the main magnet 220 and distort the main magnetic field so that the magnetic orientations of the protons of the subject 260 may vary as a function of their positions inside the gradient field, thereby encoding spatial information into MR signals generated by the region of the subject being imaged. The gradient coils 230 may include X coils (e.g., configured to generate the gradient field Gx corresponding to the X-direction), Y coils (e.g., configured to generate the gradient field Gy corresponding to the Y-direction), and/or Z coils (e.g., configured to generate the gradient field Gz corresponding to the Z-direction) (not shown in FIG. 2). The three sets of coils may generate three different magnetic fields that are used for position encoding. The gradient coils 230 may allow spatial encoding of MR signals for image reconstruction. As used herein, the X-axis, the Y-axis, the Z-axis, the X-direction, the Y-direction, and the Z-direction in the description of FIG. 2 are the same as or similar to those described in FIG. 1.

In some embodiments, the radio frequency (RF) coils 240 may be located inside the main magnet 220 and serve as transmitters, receivers, or both. For example, the RF coils 240 may be located in the detection region 270. The RF coils 240 may surround, along the Z-direction, the subject 260 that is moved into or positioned within the detection region 270. The RF coils 240 may be surrounded by the main magnet 220 and/or the gradient coils 230 around the Z-direction, and be closer to the subject 260 than the gradient coils 230. When used as transmitters, the RF coils 240 may generate RF signals that provide a third magnetic field that is utilized to generate MR signals related to the region of the subject being imaged. The third magnetic field may be perpendicular to the main magnetic field. When used as receivers, the RF coils may be responsible for detecting MR signals (e.g., echoes).

In some embodiments, the main magnet 220, the gradient coils 230, and the RF coils 240 may be circumferentially positioned with respect to the subject 260 around the z-direction. It is understood by those skilled in the art that the main magnet 220, the gradient coils 230, and the RF coils 240 may be situated in a variety of configurations around the subject.

In some embodiments, the gantry 210 may be configured to support magnets (e.g., the main magnet 220 in FIG. 2), coils (e.g., the gradient coils 230 and/or the radio frequency (RF) coils 240 in FIG. 2), etc. The gantry 210 may surround, along the Z-direction, the subject 260 that is moved into or located within the detection region 270.

In some embodiments, the patient support 250 may be configured to support the subject 260. In some embodiments, the patient support 250 may have 6 degrees of freedom, for example, three translational degrees of freedom along three coordinate directions (i.e., X-direction, Y-direction, and Z-direction) and three rotational degrees of freedom around the three coordinate directions. Accordingly, the subject 260 may be positioned by the patient support 250 within the detection region 270. Merely by way of example, the patient support 250 may move the subject 260 into the detection region 270 along the Z-direction in FIG. 1.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments of the present disclosure, the medical device 110 of the medical system 100 may include an MRI device (e.g., the MRI device in FIG. 1, the MRI device 200 in FIG. 2) and a radiation source (e.g., a linear accelerator). The MRI device may be configured to perform an imaging of a subject. The radiation source may be configured to emit a radiation beam toward the subject, for example, based on the imaging result to perform a radiation treatment of the subject.

In some embodiments, the MRI device may include a cryostat, a superconducting magnet, and a service turret. The superconducting magnet may be configured to generate a main magnetic field (e.g., the main magnetic field in FIG. 2) and disposed within the cryostat. The cryostat and the service turret may be configured to maintain the superconducting magnet in a superconducting state. In some embodiments, the superconducting magnet may have superconducting properties, for example, (essentially) a zero electrical resistance, in the superconducting state. The superconducting magnet may be in the superconducting state when the temperature of the superconducting magnet is maintained below a temperature (or referred to as a critical temperature) by, e.g., exposed in a low-temperature environment (e.g., 4.2 K).

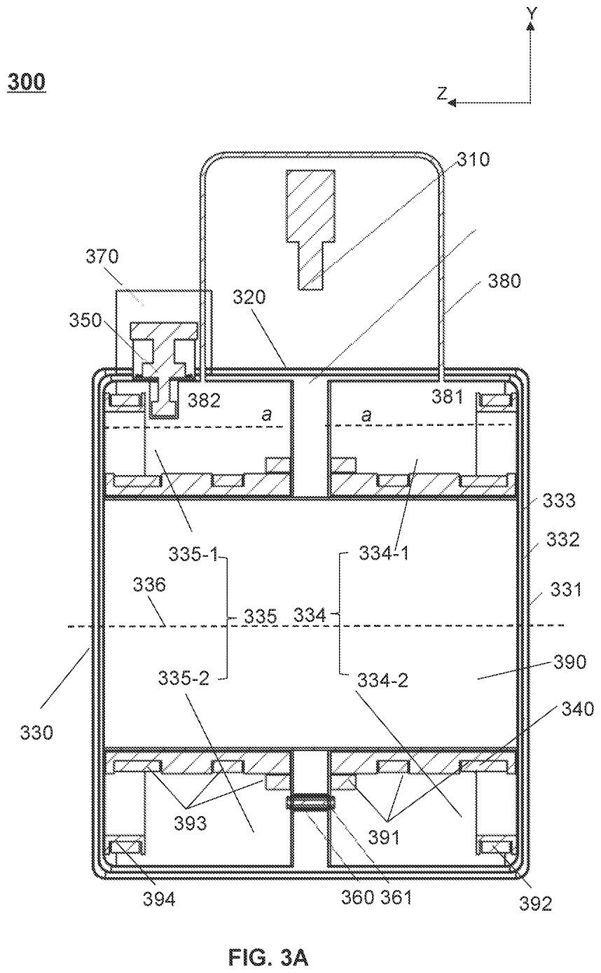

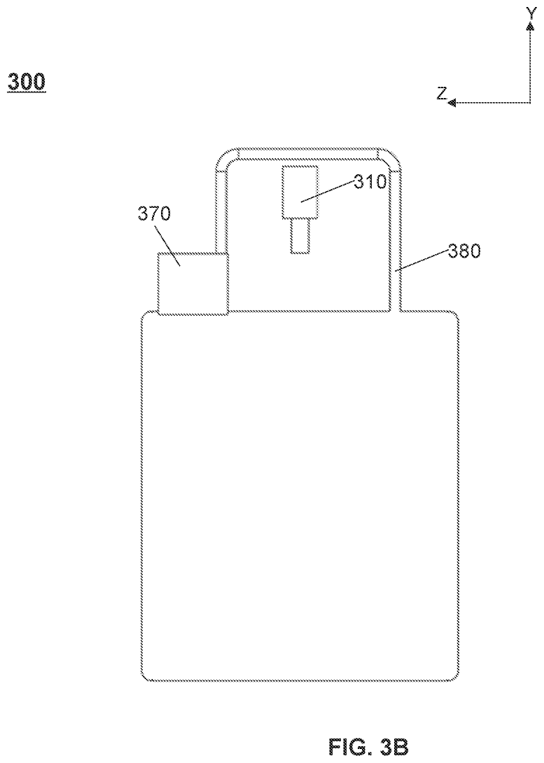

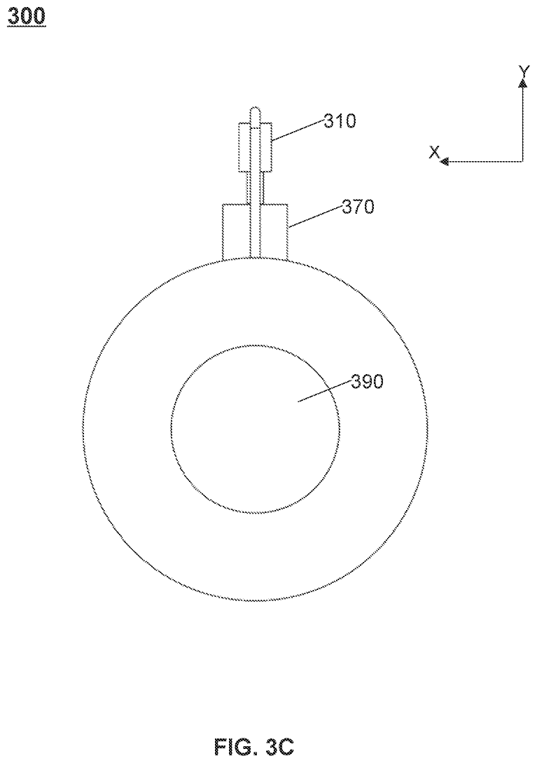

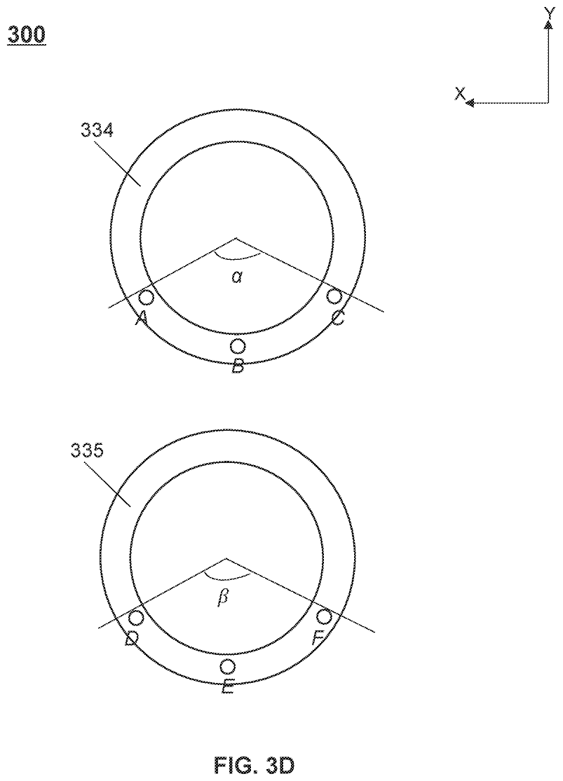

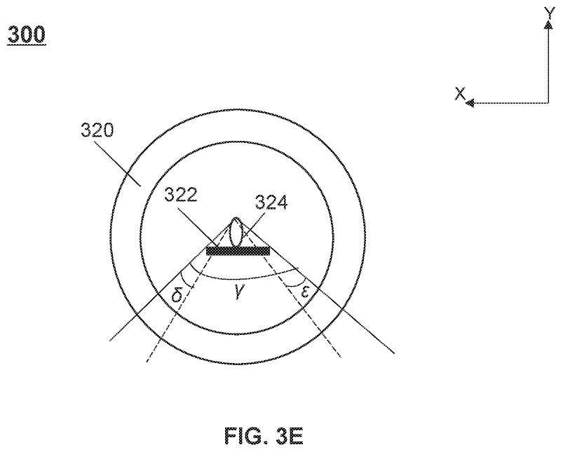

In some embodiments, the cryostat may include a first cooling chamber and a second cooling chamber. At least a portion of the first cooling chamber may be filled with a cooling medium. At least a portion of the second cooling chamber may be filled with the cooling medium. The cooling medium may include liquid helium, liquid nitrogen, water, a hyperpolarization material, etc. The cooling medium in the first cooling chamber or the second cooling chamber may be in a liquid state, a gaseous state, or a combination thereof. In some embodiments, a first portion of the first cooling chamber (e.g., a space of the first cooling chamber 334 above line a in FIG. 3A) may be filled with a gaseous-state cooling medium, and a second portion of the first cooling chamber (e.g., a space of the first cooling chamber 334 below line a in FIG. 3A) may be filled with a liquid-state cooling medium. In some embodiments, a first portion of the second cooling chamber (e.g., a space of the second cooling chamber 335 above line a in FIG. 3A) may be filled with a gaseous-state cooling medium, and a second portion of the second cooling chamber (e.g., a space of the second cooling chamber 335 below line a in FIG. 3A) may be filled with a liquid-state cooling medium. For example, the cooling medium may include gaseous-state helium and liquid-state helium.

In some embodiments, a size (e.g., a volume, an area, a width, a length) of the first cooling chamber may be the same as a size (e.g., a volume, an area, a width, a length) of the second cooling chamber. In some embodiments, the first cooling chamber and the second cooling chamber may be two separated components that are spaced apart from each other. The first cooling chamber and the second cooling chamber may be in fluid communication through a connection conduit (or referred to as a first connection conduit). The connection conduit may include a pipe. In some embodiments, a cross-section of the connection conduit may have the shape of a rectangle, an arc, a trapezoid, a triangle, a rhombus, an irregular shape, etc. For example, a length direction of the connection conduit may be parallel to a horizontal direction (e.g., the Z-direction as illustrated in FIGS. 1, 2, 3A, 3B, 4-7A, 8A, 9, and 10). As another example, a surface area of a first end of the connection conduit may be different from (e.g., larger than) a surface area of a second end of the connection conduit. It should be noted that there may be more than one connection conduit between the first cooling chamber and the second cooling chamber. For example, between the first cooling chamber and the second cooling chamber, there may be 1, 2, 3, or more connection conduits. For illustration purposes, some embodiments of the present disclosure are described with reference to one connection conduit as an example.

In some embodiments, a material of the connection conduit may be selected according to a manufacturing requirement, a manufacturing cost, etc. In some embodiments, the connection conduit may be made of a non-magnetic material or a radiation impermeable material, for example, stainless steel, tungsten, lead, iron, copper, nickel, chromium, molybdenum, or the like, or an alloy thereof. Merely by way of example, the connection conduit may include a pipe made of stainless steel.

In some embodiments, a first end of the connection conduit may be operably connected to a portion of the first cooling chamber filled with the liquid-state medium. A second end of the connection conduit may be operably connected to a portion of the second cooling chamber filled with the liquid-state medium. In some embodiments, the connection conduit may be filled with the liquid state medium.

In some embodiments, the connection conduit may be located on a side of a central axis (e.g., a central axis of the cryostat along the Z-direction, such as 336 in FIG. 3A) of the first cooling chamber and/or the second cooling chamber. In some embodiments, the cryostat, the first cooling chamber, and/or the second cooling chamber may be of an annular structure, respectively. In some embodiments, as shown in FIG. 3A, at least a portion of the first cooling chamber (e.g., 334-1 in FIG. 3A) may be above the central axis and at least a portion of the first cooling chamber (e.g., 334-2 in FIG. 3A) may be below the central axis along a first direction (e.g., the Y-direction as illustrated in FIG. 3A) from the radiation source (e.g., the radiation source 310 as illustrated in FIG. 3A) to the detection region (e.g., the detection region 270 as illustrated in FIG. 2) of the MRI device; at least a portion of the second cooling chamber (e.g., 335-1 in FIG. 3A) may be above the central axis and at least a portion of the second cooling chamber (e.g., 335-2 in FIG. 3A) may be below the central axis along the first direction. In some embodiments, the connection conduit may be located below the central axis along the first direction. A first end of the connection conduit may be located on a first side (e.g., the left side viewed from the positive X-direction) of the first cooling chamber and below the central axis. A second end of the connection conduit may be located on a second side (e.g., the right side viewed from the positive X-direction) of the second cooling chamber and below the central axis. In some embodiments, the connection conduit may be located above the central axis along the first direction. The first end of the connection conduit may be located on a first side (e.g., the left side viewed from the positive X-direction) of the first cooling chamber and above the central axis. The second end of the connection conduit may be located on a second side (e.g., the right side viewed from the positive X-direction) of the second cooling chamber and above the central axis.

In some embodiments, if the radiation beam is emitted by the radiation source when the radiation source has rotated to a position underneath the patient support (e.g., the patient support 322 in FIG. 3E) and within an angle range (e.g., angle γ in FIG. 3E), the radiation beam may first impinge on the patient support before impinging on the subject (e.g., the subject 324 in FIG. 3E), which may cause an attenuation of the radiation beam, and an amount of the radiation beam impinging on the subject to be reduced. FIG. 3E provides an illustration of the angle range (e.g., angle γ) as used herein. In order to solve the problems, the radiation beam may be emitted when the radiation source is (substantially) outside the angle range when emitting the radiation beam. In some embodiments, the first cooling chamber may have a first annular structure (e.g., shown in FIG. 3D). The first end of the connection conduit may be located at a position (e.g., position A, B, or C in FIG. 3D) within a first arc of the first annular structure. A first central angle (e.g., angle α in FIG. 3D) of the first arc may be within a first angle threshold. Exemplary first angle thresholds may be 120 degrees, 150 degrees, 180 degrees, 210 degrees, 240 degrees, etc. In some embodiments, the second cooling chamber may have a second annular structure (e.g., shown in FIG. 3D). Similar to the first end, the second end of the connection conduit may be located at a position (e.g., position D, E, or F in FIG. 3D) within a second arc of the second annular structure. A second central angle (e.g., angle β in FIG. 3D) of the second arc may be within a second angle threshold. Exemplary second angle thresholds may be 120 degrees, 150 degrees, 180 degrees, 210 degrees, 240 degrees, etc. In some embodiments, the first angle threshold or the second angle threshold may be within the angle range, such that the amount of the radiation beam traversing the connection conduit may be smaller than a first amount threshold.

In some embodiments, in order to improve the efficiency of a radiation session (e.g., shortening the duration of the treatment session), a portion of the radiation beam may be emitted when the radiation source is within a subrange of the angle range, e.g., when the radiation source is at a position within (but at or near a border of) the angle range. FIG. 3E illustrates two exemplary sub-ranges (e.g., angle δ and angle ε) at the two borders of the angle range, respectively. In some embodiments, the connection conduit may be positioned underneath the patient support and within the angle range but outside the two subranges, such that the radiation beam emitted by the radiation source when the radiation source is at a positioned within either one of the subranges may traverse the patient table but not the connection conduit to reduce or minimize the attenuation of the radiation beam by the connection conduit before impinging on the patient, or a portion thereof.

In some embodiments, the liquid-state cooling medium may transform from the liquid state to the gaseous state after absorbing heat during an operation of the system. In order to maintain the superconductivity of the superconducting magnet, the amount of the liquid-state cooling medium (before or after absorbing the heat) may need to be larger than a threshold. In some embodiments, the connection conduit may be disposed at a position (e.g., in the Y direction) below the liquid surface of the liquid-state cooling medium along the first direction when the amount of the liquid-state cooling medium is equal to or larger than the threshold. For example, the liquid surface of the liquid-state cooling medium is indicated by line a in FIG. 3A, and the connection conduit may be disposed at a position of the first cooling chamber and/or the second cooling chamber below line a. In some embodiments, a volume or a depth of the liquid-state cooling medium inside the connection conduit may be substantially constant.

In some embodiments, the radiation source may be positioned between the first cooling chamber and the second cooling chamber (e.g., the Z-direction) such that the first cooling chamber and/or the second cooling chamber may be outside the pathway (or referred to as the radiation range) in which the radiation beam emitted by the radiation source travels toward the subject, thereby reducing or avoiding the attenuation of the radiation beam by the first cooling chamber and/or the second cooling chamber. In some embodiments, the radiation source may be positioned above or (substantially) at the same level as the first cooling chamber and the second cooling chamber along the first direction (e.g., along the Y-direction), thereby reducing or avoiding the interference of the radiation source on the superconducting magnet, the main magnetic field, one or more gradient fields, and/or the MRI signals involved in the imaging using the MRI device. As used herein, “substantially,” when used to describe a property or situation A (e.g., property or situation A that the level of the first cooling chamber or the second cooling chamber is the same as the level of the radiation source along the Y-direction), indicates that the deviation from the property or situation A is below a threshold, e.g., 10%, 8%, 5%, etc.

In some embodiments, at least a portion of the radiation beam may impinge on the subject after traversing the connection conduit. In some embodiments, the volume or the depth of the liquid-state cooling medium is (substantially) constant. The attenuation of the radiation beam may be quantified, and compensated for accordingly during the radiation treatment of the subject. In some embodiments, the volume of the liquid-state cooling medium is relatively small (due to the small size of the connection conduit compared to the first cooling chamber or the second cooling chamber), an effect of the liquid-state cooling medium inside the connection conduit on the attenuation of the radiation beam may be negligible.

In some embodiments, the superconducting magnet may be housed inside the first cooling chamber and/or the second cooling chamber. In some embodiments, the cooling medium may be directly or indirectly thermally coupled with the superconducting magnet. For instance, at least a portion of the superconducting magnet may be immersed in the cooling medium (e.g., the liquid-state cooling medium). As another example, the liquid-state cooling medium may be housed in a pipeline at least a portion of which is positioned within the first cooling chamber or the second cooling chamber so that the cooling medium, without directly contacting the superconducting magnet, is thermally coupled with the superconducting magnet through the wall of the pipeline. The superconducting magnet may be cooled by the cooling medium to reduce or maintain the temperature of the superconducting magnet so as to maintain its superconducting state, thereby ensuring the performance of the superconducting magnet and reducing or avoiding the occurrence of the quenching of superconductivity.

In some embodiments, the superconducting magnet may include at least one first coil housed inside the first cooling chamber and at least one second coil housed inside the second cooling chamber. At least two of the at least one first coil and the at least one second coil may be electrically connected. In some embodiments, the at least one first coil may be symmetric with the at least one second coil. In some embodiments, the at least one first coil may include at least one first main coil and/or at least one first shield coil. The at least one second coil may include at least one second main coil and/or at least one second shield coil. It should be noted that a count of the at least one first coil and/or a count of the at least one second coil may be non-limiting, for example, 3, 4, 5, 6, etc. In some embodiments, the at least one first coil and the at least one second coil may be supported by one or more coil carriers.

In some embodiments, at least a portion of at least one superconducting wire may be housed inside the connection conduit. The at least one superconducting wire may be configured to operably connect the at least one first coil in the first cooling chamber and the at least one second coil in the second cooling chamber. In some embodiments, the connection conduit may be covered by a radiation protection component. Merely by way of example, the connection conduit may be housed inside the radiation protection component. In some embodiments, the radiation protection component may be housed inside the connection conduit. The radiation protection component may include a pipe. The radiation protection component may be configured to protect the at least a portion of the at least one superconducting wire from being exposed to the radiation beam, thereby reducing or avoiding the occurrence of the quenching of the superconductivity of the superconducting wire due to the exposure to the radiation beam. For example, the at least a portion of the at least one superconducting wire may be housed inside the radiation protection component. In some embodiments, the radiation protection component may be made of a radiation impermeable material, for example, lead, tungsten, etc., or an alloy thereof. For example, the radiation protection component may include a pipe made of lead. In some embodiments, the connection conduit and the radiation protection component may be two concentric pipes. It should be noted that the above descriptions are for illustration purposes and non-limiting. As described above, the cryostat may include multiple connection conduits. In such cases, at least a portion of at least one superconducting wire may be housed inside at least one of the multiple connection conduits.

It should be noted that the above descriptions are for illustration purposes and non-limiting. In some embodiments, the radiation protection component may be outside the connection conduit. The radiation protection component may be independent from the connection conduit, that is, the radiation protection component and the connection conduit are two separated components that are spaced apart from each other. In some embodiments, a length direction (e.g., the Z-direction as illustrated in FIGS. 1, 2, 3A, 3B, 4-7A, 8A, 9, and 10) of the radiation protection component may be parallel with a length direction of the connection conduit. The at least a portion of the at least one superconducting wire may be housed inside the radiation protection component.

In some embodiments, the first cooling chamber and the second cooling chamber may be in gas communication through a second connection conduit. The second connection conduit may include a pipe. By arranging the second connection conduit, an atmospheric pressure of the first cooling chamber may maintain the same as an atmospheric pressure of the second cooling chamber. In some embodiments, the second connection conduit may facilitate the release of pressure in the first cooling chamber and/or the second cooling chamber. In some embodiments, by employing the second connection conduit, only one of the first cooling chamber or the second cooling chamber may need to be configured with a quenching valve for pressure release (by, e.g., releasing the gaseous-state cooling medium). In some embodiments, by employing the second connection conduit, even if each of the first cooling chamber and the second cooling chamber are configured with a quenching valve for pressure release, when one quenching valve of a cooling chamber fails to function properly, the pressure buildup in that cooling chamber may be reduced or avoided because pressure may be released (by, e.g., releasing the gaseous-state cooling medium) from the quenching valve of the other cooling chamber facilitated by the second connection conduit.

In some embodiments, the second connection conduit may be made of a non-magnetic material including for example, stainless steel, a radiation impermeable material including, for example, lead, tungsten, etc., or an alloy thereof. In some embodiments, the second connection conduit may be disposed outside the pathway or the radiation range of the radiation beam in which the radiation beam travels toward the subject, thereby reducing or avoiding the attenuation of the radiation beam by the second connection conduit. It should be noted that there may be more than one second connection conduit. For example, there may be 1, 2, 3, or more second connection conduits. For illustration purposes, some embodiments of the present disclosure are described with reference to one second connection conduit.

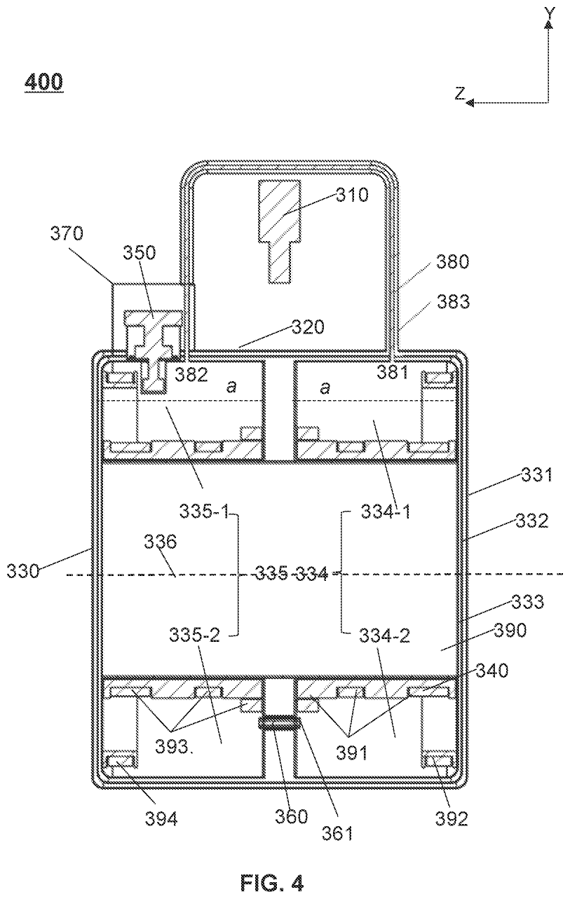

In some embodiments, the second connection conduit may be located outside the first cooling chamber and the second cooling chamber. In some embodiments, the second connection conduit may be located above the first cooling chamber and the second cooling chamber along the first direction. In some embodiments, the radiation source may be rotated within a range (also referred to as “a rotation range”) within which the radiation source emits the radiation beam to perform the radiation treatment. The second connection conduit may be positioned out of the rotation range of the radiation source. In some embodiments, at least a portion of the second connection conduit may be located above the radiation source along the first direction. In some embodiments, the second connection conduit may surround at least one side (e.g., an upper side, a left side, a right side viewed from the positive X-direction) of the radiation source. In some embodiments, a first end (e.g., 381 in FIGS. 3A, 3B, and 4) of the second connection conduit may be operably connected to an upper portion of the first cooling chamber (e.g., a portion of the first cooling chamber that is above the central axis along the first direction). The first end of the second connection conduit may be located on a first side (e.g., the right side viewed from the positive X-direction) of the radiation source. A second end (e.g., 382 in FIGS. 3A, 3B, and 4) of the second connection conduit may be operably connected to an upper portion of the second cooling chamber (e.g., a portion of the second cooling chamber that is above the central axis along the first direction). The second end of the second connection conduit may be located on a second side (e.g., the left side viewed from the positive X-direction) of the radiation source. In some embodiments, the second end of the second connection conduit may be operably connected to the upper portion of the second cooling chamber through a service turret (e.g., 370 as illustrated in FIGS. 3A, 3B, and 4).

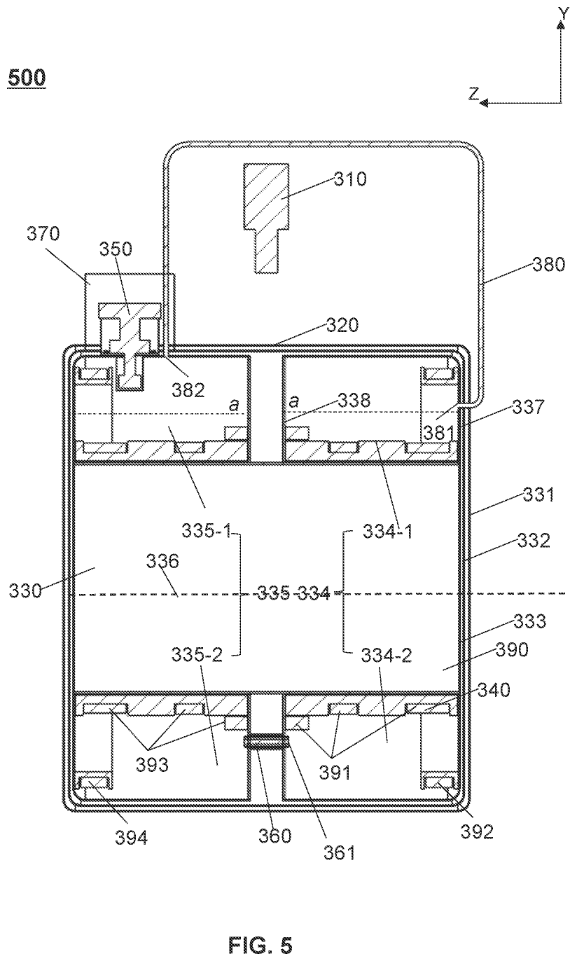

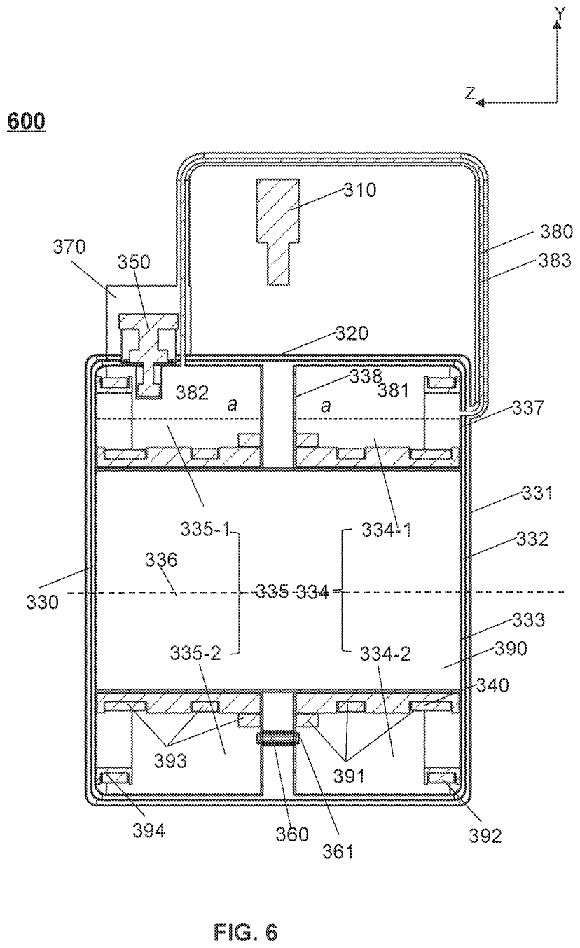

In some embodiments, a first end of the second connection conduit may be operably connected to the first cooling chamber on a far side of the first cooling chamber that is farther away from the second cooling chamber than a near side of the first cooling chamber. For instance, side 337 is a far side of the first cooling chamber 334 that is farther away from the second cooling chamber 335 than a near side 338 of the first cooling chamber 334 in FIG. 5 or FIG. 6. As described above, the first portion of the first cooling chamber (e.g., a space of the first cooling chamber 334 above line a in FIG. 3A) may be filled with the gaseous-state cooling medium and the second portion of the first cooling chamber (e.g., a space of the first cooling chamber 334 below line a in FIG. 3A) may be filled with the liquid-state cooling medium. At least a portion of the second connection conduit may be connected to the first portion of the first cooling chamber, and a remaining portion of the second connection conduit may be located above the first portion of the first cooling chamber along the first direction. For example, the first end (e.g., 381 in FIG. 5 and FIG. 6) of the second connection conduit may be operably connected to a first side (e.g., the right side viewed from the positive X-direction) of the first cooling chamber. The first end of the second connection conduit may be located at a position of the first portion of the first cooling chamber. A second end (e.g., 382 in FIG. 5 and FIG. 6) of the second connection conduit may be operably connected to an upper portion (e.g., a portion of the second cooling chamber 335 that is above the central axis 336) of the second cooling chamber. In some embodiments, the second end of the second connection conduit may be operably connected to the upper portion of the second cooling chamber through the service turret (e.g., 370 as illustrated in FIG. 5 and FIG. 6).

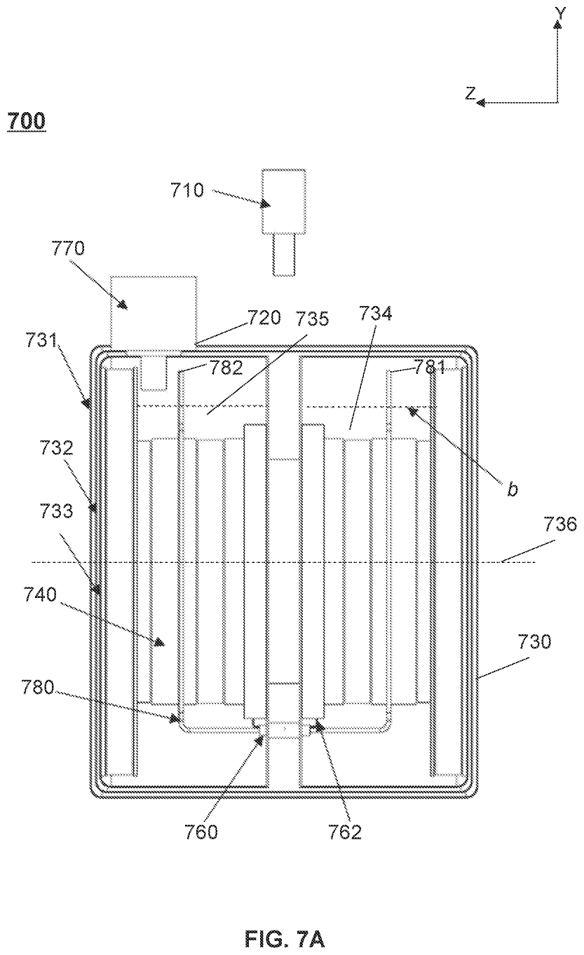

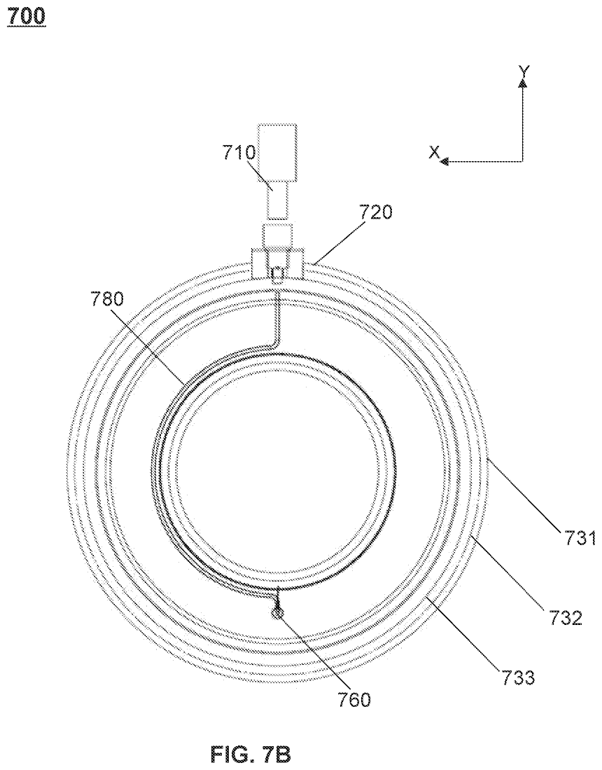

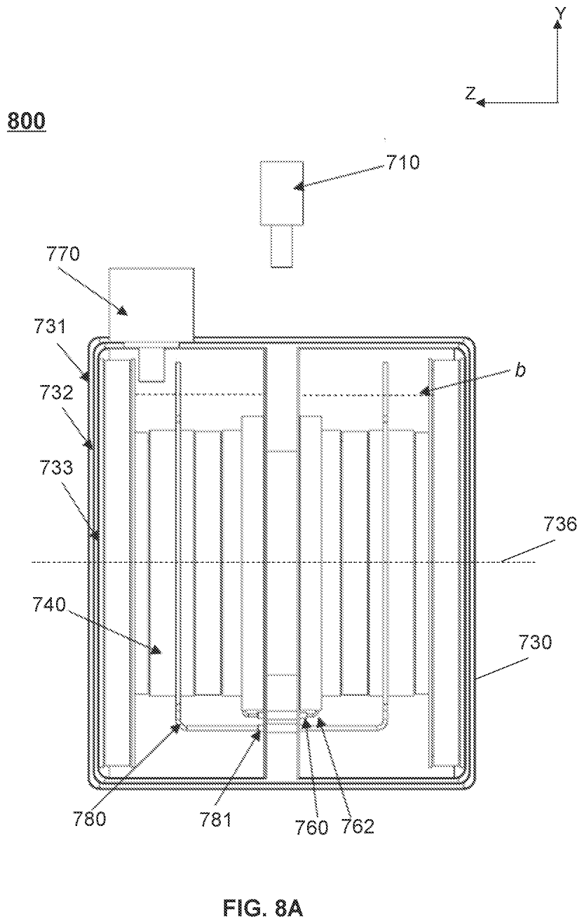

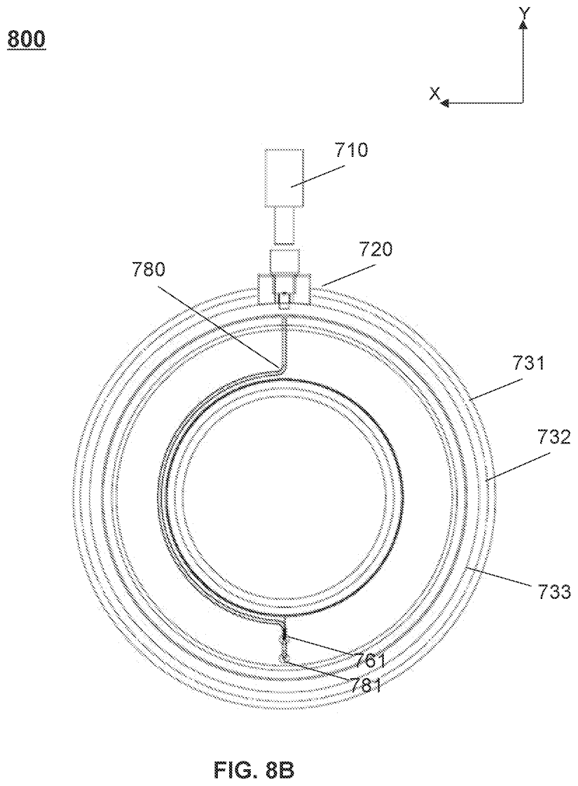

In some embodiments, at least a portion of the second connection conduit may be located inside the first portion of the first cooling chamber filled with the gaseous-state cooling medium and the first portion of the second cooling chamber filled with the gaseous-state cooling medium. A first end (e.g., 381 in FIGS. 7A and 8A) of the second connection conduit may be operably connected to the first cooling chamber at a position of the first portion of the first cooling chamber. A second end (e.g., 782 in FIGS. 7A and 8A) of the second connection conduit may be operably connected to the second cooling chamber at a position of the first portion of the second cooling chamber. In some embodiments, at least a portion of the second connection conduit may be located between at least one main coil and at least one shield coil of the superconducting magnet along a radial direction of the superconducting magnet. In some embodiments, at least a portion of the second connection conduit may be located below at least one coil (e.g., at least one main coil) of the superconducting magnet. In some embodiments, at least a portion of the second connection conduit may wrap around at least a portion of at least one coil (e.g., at least one main coil) of the supper magnet (e.g., shown in FIG. 7B). In some embodiments, as illustrated in FIGS. 7A and 8A, a section view (e.g., in the YZ-plane) of the second connection conduit may include a U shape. In some embodiments, a length direction (e.g., the Z-direction) of the connection conduit may be parallel with a length direction (e.g., the Z-direction) of the second connection conduit.

In some embodiments, at least a portion of the second connection conduit may be located inside the connection conduit. A remaining portion of the second connection conduit may be located inside the first cooling chamber and the second cooling chamber. For example, the at least a portion of the second connection conduit may be located inside the radiation protection component of the connection conduit. In some embodiments, the second connection conduit may be located outside the connection conduit. The connection conduit may be independent from the second connection conduit, that is, the connection conduit and the second connection conduit may be two separated components that are spaced apart from each other. In some embodiments, at least a portion of the second connection conduit may be housed inside a third connection conduit (e.g., a pipe). A remaining portion of the second connection conduit may be located inside the first cooling chamber and the second cooling chamber. In some embodiments, a length direction (e.g., the Z-direction) of the connection conduit may be parallel with a length direction of the third connection conduit. In some embodiments, the third connection conduit may be made of metal, for example, stainless steel, tungsten, lead, iron, copper, nickel, chromium, molybdenum, or the like, or an alloy thereof. For example, the third connection conduit may include a pipe made of stainless steel. In some embodiments, the third connection conduit may be omitted.

In some embodiments, a vacuum layer may be configured outside the second connection conduit. The vacuum layer may wrap around the second connection conduit. The vacuum layer may be configured to reduce the heat transfer between the ambient and the cooling medium (e.g., the gaseous-state cooling medium).

In some embodiments, the cryostat may include an outer vessel (e.g., an outer vacuum space (OVS)), a thermal shield, and an inner vessel (e.g., a tank). The first cooling chamber and the second cooling chamber may be housed in the inner vessel. The outer vessel may enclose the thermal shield, and the thermal shield may enclose the inner vessel 333. In some embodiments, there may be a space between the inner vessel and the outer vessel. In some embodiments, the space may house a vacuum environment. In some embodiments, the thermal shield may be disposed within the space. The outer vessel, the thermal shield, and/or the vacuum environment may be employed alone or in combination to reduce the heat transfer between the ambient and the inner vessel, as well as the first cooling chamber and the second cooling chamber within the inner vessel.

In some embodiments, the cryostat may be of an annular structure. At least one of the outer vessel, the thermal shield, or the inner vessel may also be of an annular structure. In some embodiments, the thermal shield and the outer vessel may be an integrated annular structure. In some embodiments, a recess may be positioned around at least a portion of a circumference of the integrated annular structure. For example, the recess may be configured around the entire circumference of the integrated annular structure. As another example, the recess may be configured around a portion of the circumference of the integrated annular structure.

In some embodiments, the recess may be located between the first cooling chamber and the second cooling chamber (e.g., along the Z-direction as illustrated in FIG. 1). The recess may be configured to accommodate at least a portion of the radiation source and/or provide a path for rotation of the radiation source, such that the distance between the radiation source and an axial direction of the detection region (e.g., the detection region 270 as illustrated in FIG. 2) of the MRI device may be reduced, thereby increasing the radiation dose that may reach a portion of the subject (e.g., tumor) and improving the efficacy of the radiation treatment. In some embodiments, an accelaration direction of an accelaration tube of the radiation source may be (substantially) parallel to the axial direction of the MRI device. In some embodiments, the accelartion direction of the accelaration tube of the radiation source may be (substantially) perpendicular to the axial direction of the MRI device. The acceleration tube may be configured to accelerate an electron beam to generate the treatment beam. The acceleration direction may refer to a direction along which the accelerated electron beam exits the acceleration tube.

In some embodiments, the first cooling chamber may include a first service turret. The first service turret may be mounted on an upper portion of the first cooling chamber (e.g., a portion of the first cooling chamber that is above the central axis of the first cooling chamber along the first direction). The second cooling chamber may include a second service turret different from the first service turret. The second service turret may be mounted on an upper portion of the second cooling chamber (e.g., a portion of the second cooling chamber that is above the central axis of the second cooling chamber along the first direction). In some embodiments, the first cooling chamber (e.g., the first service turret thereof) may include a first quenching valve. The second cooling chamber (e.g., the second service turret thereof) may include a second quenching valve different from the first quenching valve. The first quenching valve may be configured to facilitate releasing at least a portion of the gaseous-state cooling medium from the first cooling chamber. The second quenching valve may be configured to facilitate releasing at least a portion of the gaseous-state cooling medium from the second cooling chamber.

In some embodiments, the first cooling chamber (e.g., the first service turret thereof) and the second cooling chamber (e.g., the second service turret thereof) may share a refrigeration component (e.g., a cold head). The shared refrigeration component may be configured to cool the cooling medium that is used to cool the superconducting magnet positioned inside the first cooling chamber and the second cooling chamber. In some embodiments, the shared refrigeration component may be mounted on the first service turret. In some embodiments, the shared refrigeration component may be mounted on the second service turret.

In some embodiments, the first cooling chamber (e.g., the first service turret thereof) and the second cooling chamber (e.g., the second service turret thereof) may share a heater. The shared heater may be configured to heat the cooling medium that is used to cool the superconducting magnet positioned inside the first cooling chamber and the second cooling chamber. In some embodiments, the shared refrigeration component and the shared heater may be configured to adjust an atmospheric pressure of the first cooling chamber and/or an atmospheric pressure of the second cooling chamber.

In some embodiments, the first cooling chamber (e.g., the first service turret thereof) may include a first refrigeration component (e.g., a first cold head). The second cooling chamber (e.g., the second service turret thereof) may include a second refrigeration component (e.g., a second cold head) different from the first refrigeration component. The first refrigeration component may be configured to cool the cooling medium that is used to cool a portion of the superconducting magnet positioned inside the first cooling chamber. The second refrigeration component may be configured to cool the cooling medium that is used to cool a portion of the superconducting magnet positioned inside the second cooling chamber.

In some embodiments, the first cooling chamber (e.g., the first service turret thereof) may include a first heater. The second cooling chamber (e.g., the second service turret thereof) may include a second heater different from the first heater. The first heater may be configured to heat the cooling medium that is used to cool a portion of the superconducting magnet positioned inside the first cooling chamber. The second heater may be configured to heat the cooling medium that is used to cool a portion of the superconducting magnet positioned inside the second cooling chamber. In some embodiments, the first refrigeration component and the first heater may be configured to adjust an atmospheric pressure of the first cooling chamber. The second refrigeration component and the second heater may be configured to adjust an atmospheric pressure of the second cooling chamber.

In some embodiments, a temperature of the cooling medium and/or an atmospheric pressure of the first cooling chamber or the second cooling chamber may increase abruptly due to the quenching of the superconductivity of the superconducting magnet. In order to reduce or avoid damages to the system, at least a portion of the gaseous-state cooling medium may be released through the first quenching valve and/or the second quenching valve and/or be cooled by a refrigeration component (e.g., the shared refrigeration component, the first refrigeration component, the second refrigeration component). More descriptions of the medical device 110 may be found elsewhere in the present disclosure, for example, FIGS. 3A-10 or the descriptions thereof.

FIG. 3A provides a section view illustrating an exemplary medical device according to some embodiments of the present disclosure. FIG. 3B and FIG. 3C provide side views illustrating an exemplary medical device according to some embodiments of the present disclosure. FIG. 3D provides section views illustrating portions of an exemplary medical device according to some embodiments of the present disclosure. The medical device 300 may be an example of the medical device 110 of the medical system 100 described in FIGS. 1 and 2.

In some embodiments, the medical device 300 may include a radiation source 310 and an MRI device 320. The MRI device 320 may be configured to perform an imaging of a subject (e.g., the subject in FIG. 1, the subject 260 in FIG. 2). The radiation source 310 may be configured to emit a radiation beam toward the subject, for example, based on the imaging result to perform a radiation treatment of the subject.

In some embodiments, the MRI device 320 may include a cryostat 330, a superconducting magnet 340, and a service turret 370. The superconducting magnet 340 may be configured to generate a main magnetic field and disposed within the cryostat 330. The cryostat 330 and the service turret 370 may be configured to maintain the superconducting magnet 340 in a superconducting state.

In some embodiments, the cryostat 330 may include an outer vessel 331 (e.g., an outer vacuum space (OVS)), a thermal shield 332, and an inner vessel 333 (e.g., a tank). The outer vessel 331 may enclose the thermal shield 332, and the thermal shield 332 may enclose the inner vessel 333. In some embodiments, there may be a space between the inner vessel 333 and the outer vessel 331. In some embodiments, the space may house a vacuum environment. In some embodiments, the thermal shield 332 may be disposed within the space. The outer vessel 331, the thermal shield 332, and/or the vacuum environment may be employed alone or in combination to reduce the heat transfer between the ambient and the inner vessel 333, as well as the first cooling chamber 334 and the second cooling chamber 335 within the inner vessel 333.

In some embodiments, the cryostat 330 may be of an annular structure. At least one of the outer vessel 331, the thermal shield 332, or the inner vessel 333 may also be of an annular structure. In some embodiments, the thermal shield 332 and the outer vessel 331 may be an integrated annular structure. In some embodiments, the cryostat 330 may have a bore 390 corresponding to a detection region (e.g., the detection region 270) of the MRI device 320. In some embodiments, the outer vessel 331, the thermal shield 332, and the inner vessel 333 may be disposed coaxially (e.g., along the Z-direction) or non-coaxially.

In some embodiments, the inner vessel 333 may include a first cooling chamber 334 and a second cooling chamber 335. At least a portion of the first cooling chamber 334 may be filled with a cooling medium. At least a portion of the second cooling chamber 335 may be filled with the cooling medium. The cooling medium in the first cooling chamber 334 or the second cooling chamber 335 may be in a liquid state, a gaseous state, or a combination thereof. In some embodiments, a first portion of the first cooling chamber 334 (e.g., a space of the first cooling chamber 334 above line a in FIG. 3A) may be filled with a gaseous-state cooling medium, and a second portion of the first cooling chamber 334 (e.g., a space of the first cooling chamber 334 below line a in FIG. 3A) may be filled with a liquid-state cooling medium. In some embodiments, a first portion of the second cooling chamber 335 (e.g., a space of the second cooling chamber 335 above line a in FIG. 3A) may be filled with a gaseous-state cooling medium and a second portion of the second cooling chamber 335 (e.g., a space of the second cooling chamber 335 below line a in FIG. 3A) may be filled with a liquid-state cooling medium.

In some embodiments, the first cooling chamber 334 and the second cooling chamber 335 may be two separated components that are spaced apart from each other. The first cooling chamber 334 and the second cooling chamber 335 may be in fluid communication through a connection conduit 360. The connection conduit 360 may include a pipe. In some embodiments, the connection conduit 360 may be located below a central axis 336 of the first cooling chamber 334 and/or the second cooling chamber 335 along a first direction (e.g., the Y direction as illustrated in FIG. 3A) from the radiation source (e.g., the radiation source 310 as illustrated in FIG. 3A) to the detection region (e.g., the detection region 270 as illustrated in FIG. 2) of the MRI device 320. As shown in FIG. 3A, a portion 334-1 of the first cooling chamber 334 may be located above the central axis 336, and a portion 334-2 of the first cooling chamber 334 may be located below the central axis 336 along the first direction. A portion 335-1 of the second cooling chamber 335 may be located above the central axis 336, and a portion 335-2 of the second cooling chamber 335 may be located below the central axis 336 along the first direction. A first end of the connection conduit 360 may be located on a first side (e.g., the left side viewed from the positive X-direction) of the portion 334-2, and a second end of the connection conduit 360 may be located on a second side (e.g., the right side viewed from the positive X-direction) of the portion 335-2.

In some embodiments, the liquid-state cooling medium may transform from the liquid state to the gaseous state after absorbing heat during an operation of the medical device 300. In order to maintain the superconductivity of the superconducting magnet 340, the amount of the liquid-state cooling medium (before or after absorbing the heat) may need be larger than a threshold. In some embodiments, the connection conduit 360 may be disposed at a position (along the Y-direction) below the liquid surface of the liquid-state cooling medium when the amount of the liquid-state cooling medium is equal to or larger than the threshold. For example, the liquid surface of the liquid-state cooling medium is indicated by line a in FIG. 3A, the connection conduit 360 may be disposed at a position of the first cooling chamber 334 and/or the second cooling chamber 335 below line a. In some embodiments, a volume or a depth of the liquid-state cooling medium inside the connection conduit 360 may be substantially constant.

In some embodiments, the radiation source 310 may be positioned above or (substantially) at the same level as the first cooling chamber 334 and the second cooling chamber 335 along the first direction (e.g., along the Y-direction), thereby reducing or avoiding the interference of the radiation source 310 on the superconducting magnet 340, the main magnetic field, one or more gradient fields, and/or the MRI signals involved in the imaging using the MRI device 320. As used herein, “substantially,” when used to describe a property or situation A (e.g., property or situation A that the level of the first cooling chamber 334 or the second cooling chamber 335 is the same as the level of the radiation source 310 along the first direction), indicates that the deviation from the property or situation A is below a threshold, e.g., 10%, 8%, 5%, etc. In some embodiments, along a second direction (e.g., the Z-direction) perpendicular to the first direction, the radiation source 310 may be positioned between the first cooling chamber 334 and the second cooling chamber 335 such that the first cooling chamber 334 and/or the second cooling chamber 335 may be outside the pathway (or referred to as the radiation range) in which the radiation beam emitted by the radiation source 310 travels toward the subject, thereby reducing or avoiding the attenuation of the radiation beam by the first cooling chamber 334 and/or the second cooling chamber 335.

In some embodiments, at least a portion of the radiation beam may impinge on the subject after traversing the connection conduit 360. In some embodiments, the volume or the depth of the liquid-state cooling medium is (substantially) constant. The attenuation of the radiation beam may be quantified, and compensated for accordingly during a radiation treatment of the subject. In some embodiments, the volume of the liquid-state cooling medium is relatively small (due to the small size of the connection conduit 360 compared to the first cooling chamber 334 or the second cooling chamber 335), an effect of the liquid-state cooling medium inside the connection conduit 360 on the attenuation of the radiation beam may be negligible.

In some embodiments, the superconducting magnet 340 may be housed inside the first cooling chamber 334 and/or the second cooling chamber 335. In some embodiments, the superconducting magnet 340 may include at least one first coil (e.g., rectangles inside the first cooling chamber 334 in FIG. 3A) housed inside the first cooling chamber 334 and at least one second coil (e.g., rectangles inside the second cooling chamber 335 in FIG. 3B) housed inside the second cooling chamber 335. The at least one first coil may include three main coils 391 and a shield coil 392. The at least one second coil may include three main coils 393 and a shield coil 394. It should be noted that a count of the at least one first coil and/or a count of the at least one second coil may be non-limiting, for example, 3, 4, 5, 6, etc.

In some embodiments, at least a portion of at least one superconducting wire may be housed inside the connection conduit 360. The at least one superconducting wire may be configured to operably connect the at least one first coil in the first cooling chamber 334 and the at least one second coil in the second cooling chamber 335. In some embodiments, a radiation protection component 361 may be housed inside the connection conduit 360. The radiation protection component 361 may include a pipe. The radiation protection component 361 may be configured to protect the at least a portion of the at least one superconducting wire from being exposed to the radiation beam. In some embodiments, the radiation protection component 361 may be made of a radiation impermeable material, for example, lead, tungsten, etc., or an alloy thereof. For example, the radiation protection component 361 may include a pipe made of lead. In some embodiments, the connection conduit 360 and the radiation protection component 361 may be two concentric pipes.

In some embodiments, the first cooling chamber 334 and the second cooling chamber 335 may be in gas communication through a second connection conduit 380. The second connection conduit 380 may include a pipe. By arranging the second connection conduit 380, an atmospheric pressure of the first cooling chamber 334 may be controllable to be the same as an atmospheric pressure of the second cooling chamber 335. In some embodiments, the second connection conduit 380 may facilitate the release of pressure in the first cooling chamber 334 and/or the second cooling chamber 335.

In some embodiments, the second connection conduit 380 may be disposed outside the pathway or the radiation range of the radiation beam in which the radiation beam travels toward the subject, thereby reducing or avoiding the attenuation of the radiation beam by the second connection conduit 380. In some embodiments, the second connection conduit 380 may be located outside the first cooling chamber 334 and the second cooling chamber 335. At least a portion of the second connection conduit 380 may be located above the radiation source 310 along the first direction. In some embodiments, the second connection conduit 380 may surround at least one side (e.g., an upper side, a left side, a right side) of the radiation source 310. In some embodiments, a first end 381 of the second connection conduit 380 may be operably connected to an upper portion (e.g., the portion 334-1) of the first cooling chamber 334. The first end 381 of the second connection conduit 380 may be located on a first side (e.g., the right side viewed from the positive X-direction) of the radiation source 310. A second end 382 of the second connection conduit 380 may be operably connected to an upper portion (e.g., the portion 335-1) of the second cooling chamber 335. The second end 382 of the second connection conduit 380 may be located on a second side (e.g., the left side viewed from the positive X-direction) of the radiation source 310. In some embodiments, the second end 382 of the second connection conduit 380 may be operably connected to an upper portion of the second cooling chamber 335 through the service turret 370.

In some embodiments, the thermal shield 332 may be a shielding cover having a low surface emissivity, which may effectively reduce the heat transfer between the outer vessel 331 and the inner vessel 333, thereby reducing the amount of evaporation of the liquid-state cooling medium in the inner vessel 333 and reducing or avoiding the occurrence of the quenching of superconductivity of the superconducting magnet 340.

In some embodiments, the outer vessel 331 may be made of a metal, for example, carbon steel, stainless steel, etc., or an alloy thereof. In some embodiments, the inner vessel 333 may be made of a metal, for example, carbon steel or stainless steel, etc., or an alloy thereof.

In some embodiments, the service turret 370 may be operably connected with the outer vessel 331. At least a portion of the service turret 370 may protrude outside the outer vessel 331. The service turret 370 may include an accommodation space that is in fluid communication with the outer vessel 331. In some embodiments, the service turret 370 may include a refrigeration component 350 and a quenching valve (not shown in FIG. 3A). In some embodiments, at least a portion of the refrigeration component 350 may be disposed in the outer vessel 331. The refrigeration component 350 may be configured to cool the thermal shield 332 to a specific temperature (e.g., 30-50K) and/or cool the superconducting magnet 340 to a superconducting state. In some embodiments, the refrigeration component 350 may include a first-stage cold head and/or a second-stage cold head. The first stage cold head may be configured to cool the superconducting magnet 340 to the superconducting state. The second stage cold head may be configured to cool the thermal shield 332 to the specific temperature (e.g., 30-50K).

It should be noted that the above descriptions are for illustration purposes and non-limiting. In some embodiments, the radiation protection component 361 may be outside the connection conduit 360. The radiation protection component 361 may be independent from the connection conduit 360, that is, the radiation protection component 361 and the connection conduit 360 are two separated components that are spaced apart from each other. In some embodiments, a length direction (e.g., the Z-direction as illustrated in FIGS. 1, 2, 3A, 3B, 4-7A, 8A, 9, and 10) of the radiation protection component 361 may be parallel with a length direction of the connection conduit 360. The at least a portion of the at least one superconducting wire may be housed inside the radiation protection component 361. More descriptions of the medical device 300 may be found elsewhere in the present disclosure, for example, the descriptions of the medical device 110 in FIGS. 1 and 2.

FIG. 4 provides a section view illustrating an exemplary medical device according to some embodiments of the present disclosure. The medical device 400 may be an example of the medical device 110 of the medical system 100 described in FIGS. 1 and 2. In some embodiments, the medical device 400 may be similar to the medical device 300 except that the medical device 400 further includes a vacuum layer 383.

The vacuum layer 383 may be maintained outside the second connection conduit 380 in FIG. 4. The vacuum layer 383 may wrap around the second connection conduit 380. The vacuum layer 383 may be configured to reduce the heat transfer between the ambient and the cooling medium. More descriptions of the medical device 400 may be found elsewhere in the present disclosure, for example, the descriptions of the medical device 110 described in FIGS. 1 and 2, the medical device 300 in FIGS. 3A-3D, or the descriptions thereof.

FIG. 5 provides a section view illustrating an exemplary medical device according to some embodiments of the present disclosure. The medical device 500 may be an example of the medical device 110 of the medical system 100 described in FIGS. 1 and 2. In some embodiments, the medical device 500 may be similar to the medical device 300 except that a position of the second connection conduit 380 is different.

Different from the second connection conduit 380 in FIG. 3A, the first end 381 of the second connection conduit 380 in FIG. 5 may be operably connected to the first cooling chamber 334 on a far side 337 of the first cooling chamber 334 that is farther away from the second cooling chamber 335 than a near side 338 of the first cooling chamber 334. In some embodiments, a first portion of the first cooling chamber 334 (e.g., a space of the first cooling chamber 334 above line a) may be filled with a gaseous-state cooling medium and a second portion of the first cooling chamber 334 (e.g., a space of the first cooling chamber 334 below line a) may be filled with the liquid-state cooling medium. At least a portion of the second connection conduit 380 may be connected to the first portion of the first cooling chamber 334, and a remaining portion of the second connection conduit 380 may be located above the first portion of the first cooling chamber 334 along the first direction (the Y-direction as illustrated in FIG. 5). For example, the first end 381 of the second connection conduit 380 may be operably connected to a side (e.g., the right side viewed from the positive X-direction) of the first cooling chamber 334. The first end 381 of the second connection conduit 380 may be located at a position of the first portion of the first cooling chamber 334. The first end 381 of the second connection conduit 380 may be located on a first side of the radiation source 310 (e.g., the right side viewed from the positive X-direction). In some embodiments, the second end 382 of the second connection conduit 380 in FIG. 5 may be operably connected to an upper portion of the second cooling chamber 335 that is above the central axis 336 of the second cooling chamber 335. The second end 382 of the second connection conduit may be located on a second side (e.g., the left side viewed from the positive X-direction) of the radiation source 310. More descriptions of the medical device 500 may be found elsewhere in the present disclosure, for example, the descriptions of the medical device 110 described in FIGS. 1 and 2, the medical device 300 in FIGS. 3A-3D, or the descriptions thereof.

FIG. 6 provides a section view illustrating an exemplary medical device according to some embodiments of the present disclosure. The medical device 600 may be an example of the medical device 110 of the medical system 100 described in FIGS. 1 and 2. In some embodiments, the medical device 600 may be similar to the medical device 500 except that the medical device 600 further includes the vacuum layer 383.

The vacuum layer 383 may be maintained outside the second connection conduit 380 in FIG. 6. The vacuum layer 383 may wrap around the second connection conduit 380. The vacuum layer 383 may be configured to reduce the heat transfer between the ambient and the cooling medium. More descriptions of the medical device 600 may be found elsewhere in the present disclosure, for example, the descriptions of the medical device 110 described in FIGS. 1 and 2, FIGS. 3A-3D and 5, or the descriptions thereof.

FIG. 7A and FIG. 7B provide section views illustrating an exemplary medical device according to some embodiments of the present disclosure. The medical device 700 may be an example of the medical device 110 of the medical system 100 described in FIGS. 1 and 2.

As shown in FIG. 7A and FIG. 7B, the medical device 700 may include a radiation source 710 and an MRI device 720. The MRI device 720 may be configured to perform an imaging of a subject (e.g., the subject in FIG. 1, the subject 260 in FIG. 2). The radiation source 710 may be configured to emit a radiation beam toward the subject, for example, based on the imaging result to perform a radiation treatment of the subject.

In some embodiments, the MRI device 720 may include a cryostat 730, a superconducting magnet 740, and a service turret 770. In some embodiments, the cryostat 730 may include an outer vessel 731 (e.g., an outer vacuum space (OVS)), a thermal shield 732, and an inner vessel 733 (e.g., a tank). In some embodiments, the service turret 770 may include a quenching valve, a refrigeration component (e.g., a cold head), etc.

In some embodiments, the inner vessel 733 may include a first cooling chamber 734 and a second cooling chamber 735. At least a portion of the first cooling chamber 734 may be filled with a cooling medium. At least a portion of the second cooling chamber 735 may be filled with the cooling medium. In some embodiments, a first portion of the first cooling chamber 734 (e.g., a space of the first cooling chamber 734 above line b in FIG. 7A) may be filled with a gaseous-state cooling medium, and a second portion of the first cooling chamber 734 (e.g., a space of the first cooling chamber 734 below line b in FIG. 7A) may be filled with a liquid-state cooling medium. In some embodiments, a first portion of the second cooling chamber 735 (e.g., a space of the second cooling chamber 735 above line b in FIG. 7A) may be filled with a gaseous-state cooling medium, and a second portion of the second cooling chamber 735 (e.g., a space of the second cooling chamber 735 below line b in FIG. 7A) may be filled with a liquid-state cooling medium.

In some embodiments, the first cooling chamber 734 and the second cooling chamber 735 may be in fluid communication through a connection conduit 760 (e.g., a pipe). In some embodiments, the connection conduit 760 may be located below a central axis 736 of the first cooling chamber 734 and/or the second cooling chamber 735.

In some embodiments, the superconducting magnet 740 may be housed inside the first cooling chamber 734 and the second cooling chamber 735. In some embodiments, the superconducting magnet 740 may include at least one first coil housed inside the first cooling chamber 734 and at least one second coil housed inside the second cooling chamber 735. In some embodiments, the at least one first coil may include at least one first main coil and/or at least one first shield coil. The at least one second coil may include at least one second main coil and/or at least one second shield coil. It should be noted that a count of the at least one first coil and/or the at least one second coil may be non-limiting, for example, 3, 4, 5, 6, etc. In some embodiments, the connection conduit 760 may be located below at least one coil (e.g., the at least one first main coil, the at least one second main coil) of the superconducting magnet 740 (e.g., along the Y-direction).

In some embodiments, at least a portion of at least one superconducting wire 762 may be housed inside the connection conduit 760. The at least one superconducting wire 762 may be configured to operably connect the at least one first coil in the first cooling chamber 734 and the at least one second coil in the second cooling chamber 735. In some embodiments, a radiation protection component (not shown in FIG. 7A and FIG. 7B) may be housed inside the connection conduit 760. The radiation protection component may include a pipe. The radiation protection component may be configured to protect the at least a portion of the at least one superconducting wire 762 from being exposed to the radiation beam. In some embodiments, the at least a portion of at least one superconducting wire 762 may be housed inside the radiation protection component.

In some embodiments, the first cooling chamber 734 and the second cooling chamber 735 may be in gas communication through a second connection conduit 780 (e.g., a pipe). By arranging the second connection conduit 780, an atmospheric pressure of the first cooling chamber 734 may be controllable to be the same as an atmospheric pressure of the second cooling chamber 735. In some embodiments, the second connection conduit 780 may facilitate the release of pressure in the first cooling chamber 734 and/or the second cooling chamber 735.

In some embodiments, at least a portion of the second connection conduit 780 may be inside the first portion of the first cooling chamber 734 and the first portion of the second cooling chamber 735. A first end of the second connection conduit 780 may be operably connected to the first cooling chamber 734 at a position of the first portion of the first cooling chamber 734. A second end 782 of the second connection conduit 780 may be operably connected to the second cooling chamber 735 at a position of the first portion of the second cooling chamber 735.

In some embodiments, at least a portion of the second connection conduit 780 may be located below at least one coil (e.g., the at least one first main coil, the at least one second main coil) of the superconducting magnet 740 along a first direction from the radiation source 710 to a detection region (the detection region 270 as illustrated in FIG. 2) of the MRI device 720. In some embodiments, a length direction (e.g., the Z-direction) of the connection conduit 760 may be parallel with a length direction (e.g., the Z-direction) of the second connection conduit 780. In some embodiments, a section view of the second connection conduit 780 may include a U shape. In some embodiments, at least a portion of the second connection conduit 780 may wrap around at least a portion of at least one coil (e.g., the at least one first main coil, the at least one second main coil) of the superconducting magnet 740. In some embodiments, at least a portion of the second connection conduit 780 may be located inside the connection conduit 760. For example, the at least a portion of the second connection conduit 780 may be located inside the radiation protection component of the connection conduit 760, thereby avoiding the radiation beam impinging on the second connection conduit 780. In some embodiments, the cryostat 730 and the superconducting magnet 740 may be the same as or similar to the cryostat 330 and the superconducting magnet 340, respectively. More descriptions of the medical device 700 may be found elsewhere in the present disclosure, for example, the descriptions of the medical device 110 described in FIGS. 1 and 2, the medical device 300 in FIGS. 3A-3D, or the descriptions thereof.

FIGS. 8A and 8B provide a section view illustrating an exemplary medical device according to some embodiments of the present disclosure. The medical device 800 may be an example of the medical device 110 of the medical system 100 described in FIGS. 1 and 2. In some embodiments, the medical device 800 may be similar to the medical device 700 except that a position of the second connection conduit 780 is different.

Different from the second connection conduit 780 in FIG. 7A and FIG. 7B, the second connection conduit 780 in FIGS. 8A and 8B may be located outside the connection conduit 760. The second connection conduit 780 may be independent from the connection conduit 760, that is, the connection conduit 760 and the second connection conduit 780 may be two separated components that are spaced apart from each other. In some embodiments, at least a portion of the second connection conduit 780 may be housed inside a third connection conduit 781. The third connection conduit 781 may include a pipe. A remaining portion of the second connection conduit 780 may be located inside the first cooling chamber 734 and the second cooling chamber 735. In some embodiments, a length direction (e.g., the Z-direction) of the connection conduit 760 may be parallel with a length direction of the third connection conduit 781. More descriptions of the medical device 800 may be found elsewhere in the present disclosure, for example, the descriptions of the medical device 110 in FIGS. 1 and 2, the medical device 300 in FIGS. 3A-3D, the medical device 700 in FIG. 7A and FIG. 7B, or the descriptions thereof.

It should be noted that the above descriptions are for illustration purposes and non-limiting. The medical device 700 or 800 may include two connection conduits configured to facilitate the gas communication between the first cooling chamber 734 and the second cooling chamber 735. The two connection conduits may include the connection conduit 780 and a connection conduit similar to or the same as the connection conduit 380 in FIGS. 3A-6.

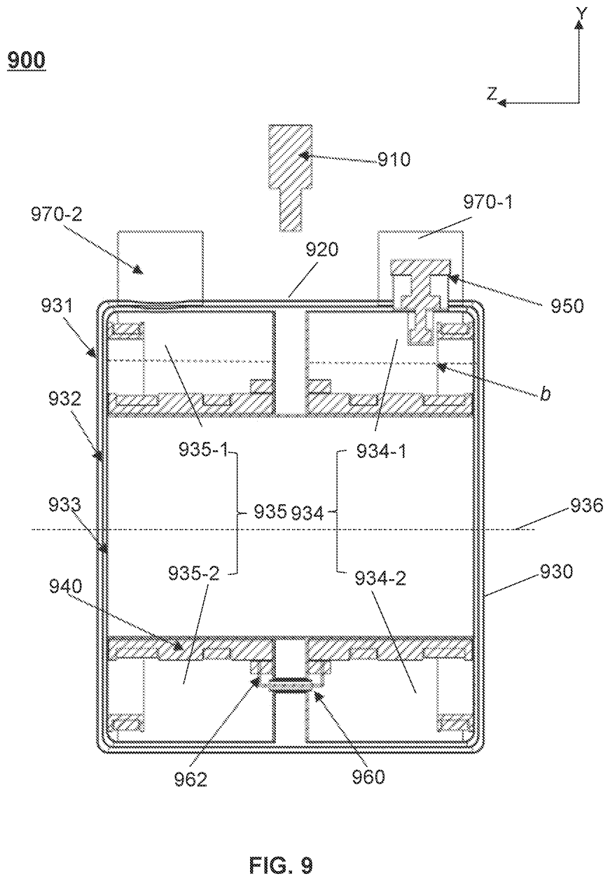

FIG. 9 provides a section view illustrating an exemplary medical device according to some embodiments of the present disclosure. The medical device 900 may be an example of the medical device 110 of the medical system 100 described in FIGS. 1 and 2.

As shown in FIG. 9, the medical device 900 may include a radiation source 910 and an MRI device 920. The MRI device 920 may be configured to perform an imaging of a subject (e.g., the subject in FIG. 1, the subject 260 in FIG. 2). The radiation source 910 may be configured to emit a radiation beam toward the subject, for example, based on the imaging result to perform a radiation treatment of the subject.

As shown in FIG. 9, the MRI device 920 may include a cryostat 930, a superconducting magnet 940, a first service turret 970-1, and a second service turret 970-2. In some embodiments, the cryostat 930 may include an outer vessel 931 (e.g., an outer vacuum space (OVS)), a thermal shield 932, and an inner vessel 933 (e.g., a tank).

The inner vessel 933 may include a first cooling chamber 934 and a second cooling chamber 935. At least a portion of the first cooling chamber 934 may be filled with a cooling medium. At least a portion of the second cooling chamber 935 may be filled with the cooling medium. In some embodiments, a portion of the first cooling chamber 934 (e.g., a space of the first cooling chamber 934 above line c in FIG. 9) may be filled with a gaseous-state cooling medium, and a portion of the first cooling chamber 934 (e.g., a space of the first cooling chamber 934 below line c in FIG. 9) may be filled with a liquid-state cooling medium. In some embodiments, a portion of the second cooling chamber 935 (e.g., a space of the second cooling chamber 935 above line c in FIG. 9) may be filled with a gaseous-state cooling medium, and a portion of the second cooling chamber 935 (e.g., a space of the second cooling chamber 935 below line c in FIG. 9) may be filled with a liquid-state cooling medium.

In some embodiments, the first cooling chamber 934 and the second cooling chamber 935 may be in fluid communication through a connection conduit 960. The connection conduit 960 may include a pipe. In some embodiments, the connection conduit 960 may be located below a central axis 936 of the first cooling chamber 934 and/or the second cooling chamber 935. As shown in FIG. 9, a portion 934-1 of the first cooling chamber 934 may be located above the central axis 936, and a portion 934-2 of the first cooling chamber 934 may be located below the central axis 936. A portion 935-1 of the second cooling chamber 935 may be located above the central axis 936, and a portion 935-2 of the second cooling chamber 935 may be located below the central axis 936. A first end of the connection conduit 960 may be located on a first side (e.g., the left side viewed from the positive X-direction) of the portion 934-2 of the first cooling chamber 934. A second end of the connection conduit 960 may be located on a second side (e.g., the right side viewed from the positive X-direction) of the portion 935-2 of the second cooling chamber 935.

In some embodiments, the superconducting magnet 940 may be housed inside the first cooling chamber 934 and the second cooling chamber 935. In some embodiments, the superconducting magnet 940 may include at least one first coil (e.g., rectangles inside the first cooling chamber 934 in FIG. 9) housed inside the first cooling chamber 934 and at least one second coil (e.g., rectangles inside the second cooling chamber 935 in FIG. 9) housed inside the second cooling chamber 935. It should be noted that a count of the at least one first coil and/or the at least one second coil may be non-limiting, for example, 3, 4, 5, 6, etc.

In some embodiments, at least a portion of at least one superconducting wire 962 may be housed inside the connection conduit 960. The at least one superconducting wire 962 may be configured to operably connect the at least one first coil in the first cooling chamber 934 and the at least one second coil in the second cooling chamber 935. In some embodiments, a radiation protection component (not shown in FIG. 9) may be housed inside the connection conduit 960. The radiation protection component may include a pipe. The radiation protection component may be configured to protect the at least a portion of the at least one superconducting wire 962 from being exposed to the radiation beam.

In some embodiments, the first service turret 960-1 may be mounted on the first cooling chamber 934 (e.g., the portion 934-1 thereof). The second service turret 960-2 may be mounted on the second cooling chamber 935 (e.g., the portion 935-1 thereof). In some embodiments, the first cooling chamber 934 (e.g., the first service turret 960-1 thereof) may include a first quenching valve (not shown in FIG. 9). The second cooling chamber 935 (e.g., the second service turret 960-2 thereof) may include a second quenching valve (not shown in FIG. 9) different from the first quenching valve. The first quenching valve may be configured to facilitate releasing at least a portion of the gaseous-state cooling medium from the first cooling chamber 934. The second quenching valve may be configured to facilitate releasing at least a portion of the gaseous-state cooling medium from the second cooling chamber 935.

As shown in FIG. 9, the first cooling chamber 934 (e.g., the first service turret 960-1 thereof) may include a refrigeration component 950 (e.g., a cold head). The refrigeration component 950 may be configured to cool the cooling medium that is used to cool the superconducting magnet 940 positioned inside the first cooling chamber 934 and the second cooling chamber 935. In some embodiments, the cryostat 930 and the superconducting magnet 940 may be the same as or similar to the cryostat 330 and the superconducting magnet 340, respectively. More descriptions of the medical device 900 may be found elsewhere in the present disclosure, for example, the descriptions of the medical device 110 described in FIG. 1 and FIG. 2, the medical device 300 in FIGS. 3A-3D, or the descriptions thereof.

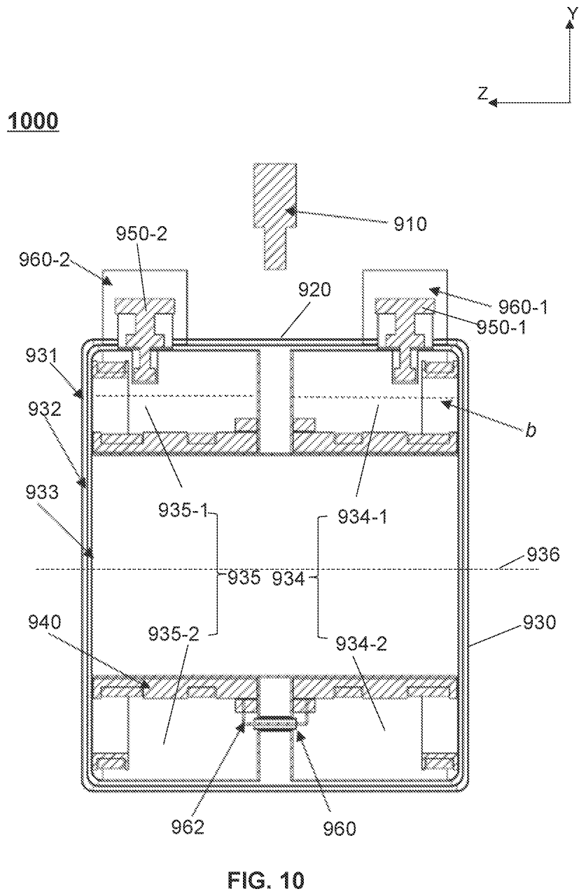

FIG. 10 provides a section view illustrating an exemplary medical device according to some embodiments of the present disclosure. The medical device 1000 may be an example of the medical device of the medical system 100 described in FIGS. 1 and 2. In some embodiments, the medical device 1000 may be similar to the medical device 900 except that a structure of the first service turret 970-1 and the second service turret 970-2 is different.

Different from the first service turret 970-1 and the second service turret 970-2 in FIG. 9, the first service turret 960-1 in FIG. 10 may include a first quenching valve (not shown in FIG. 10) and a first refrigeration component 950-1 (e.g., a first cold head), and the second service turret 960-2 in FIG. 10 may include a second quenching valve (not shown in FIG. 10) different from the first quenching valve and a second refrigeration component 950-2 (e.g., a second cold head) different from the first refrigeration component 950-1. The first quenching valve may be configured to facilitate releasing at least a portion of a gaseous-state cooling medium from the first cooling chamber 934. The second quenching valve may be configured to facilitate releasing at least a portion of a gaseous-state cooling medium from the second cooling chamber 935. The first refrigeration component 950-1 may be configured to cool the cooling medium that is used to cool a portion of the superconducting magnet 940 positioned inside the first cooling chamber 934. The second refrigeration component 950-2 may be configured to cool the cooling medium that is used to cool a portion of the superconducting magnet 940 positioned inside the second cooling chamber 935. More descriptions of the medical device 1000 may be found elsewhere in the present disclosure, for example, the descriptions of the medical device 110 described in FIGS. 1 and 2, FIGS. 3A-3D and 9, or the descriptions thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including a subject oriented programming language such as Java, Scala, Smalltalk, Eiffel, JADE, Emerald, C++, C #, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system comprising:
a magnetic resonance imaging (MRI) device configured to perform an imaging of a subject, wherein
the MRI device includes a cryostat;
the cryostat includes a first cooling chamber and a second cooling chamber that are in fluid communication through a connection conduit; and
the connection conduit is located on a side of a central axis of the first cooling chamber or a central axis of the second cooling chamber; and
a radiation source configured to emit a radiation beam toward the subject, the radiation source being positioned between the first cooling chamber and the second cooling chamber such that the first cooling chamber and the second cooling chamber are outside of a radiation range of the radiation beam.

2. The system of claim 1, wherein the connection conduit is filled with in a liquid-state cooling medium filled in a portion of the first cooling chamber and a portion of the second cooling chamber.

3. The system of claim 1, wherein the connection conduit is located below the central axis of the first cooling chamber or the central axis of the second cooling chamber.

4. The system of claim 1, wherein
the first cooling chamber has a first annular structure;
a first end of the connection conduit is located at a position within a first arc of the first annular structure;
the second cooling chamber has a second annular structure; and
a second end of the connection conduit is located at a position within a second arc of the second annular structure.

5. The system of claim 1, wherein
at least one superconducting wire is housed inside the connection conduit;
at least one first coil is housed inside the first cooling chamber;
at least one second coil is housed inside the second cooling chamber; and
the at least one superconducting wire is configured to operably connect the at least one first coil and the at least one second coil.

6-7. (canceled)

8. The system of claim 1, wherein the connection conduit includes a pipe.

9. The system of claim 1, wherein the first cooling chamber and the second cooling chamber are in gas communication through a second connection conduit.

10. The system of claim 9, wherein the second connection conduit is located outside the first cooling chamber and the second cooling chamber.

11-13. (canceled)

14. The system of claim 9, further including a vacuum layer housed outside the second connection conduit.

15. The system of claim 9, wherein
a first end of the second connection conduit is located inside a portion of the first cooling chamber filled with a gaseous-state cooling medium, and
a second end of the second connection conduit is located inside a portion of the second cooling chamber filled with the gaseous-state cooling medium.

16. The system of claim 15, wherein a length direction of the connection conduit is parallel with a length direction of the second connection conduit.

17. The system of claim 15, wherein the second connection conduit is located inside the connection conduit.

18. The system of claim 9, wherein the connection conduit is independent from the second connection conduit.

19. The system of claim 1, wherein
the connection conduit is made of metal, or
the second connection conduit is made of stainless steel or a radiation impermeable material.

20. (canceled)

21. The system of claim 1, wherein:
the first cooling chamber includes a first quenching valve; and
the second cooling chamber includes a second quenching valve different from the first quenching valve.

22. The system of claim 1, wherein
the first cooling chamber and the second cooling chamber share a cold head.

23. The system of claim 1, wherein:
the first cooling chamber includes a first cold head; and
the second cooling chamber includes a second cold head different from the first cold head.

24. The system of claim 1, wherein
at least a portion of the first cooling chamber is filled with a cooling medium; or
at least a portion of the second cooling chamber is filled with the cooling medium.

25. (canceled)

26. A system comprising:
a magnetic resonance imaging (MRI) device configured to perform an imaging of a subject, wherein
the MRI device includes a cryostat;
the cryostat includes a first cooling chamber and a second cooling chamber; and
the first cooling chamber and the second cooling chamber are in gas communication through a connection conduit; and
a radiation source configured to emit a radiation beam toward the subject, the radiation source being positioned between the first cooling chamber and the second cooling chamber such that the first cooling chamber and the second cooling chamber are outside of a radiation range of the radiation beam, wherein
the connection conduit is positioned out of a rotation range of the radiation source.

27-30. (canceled)

31. A system comprising:
a magnetic resonance imaging (MRI) device configured to perform an imaging of a subject, wherein
the MRI device includes a cryostat;
the cryostat includes a first cooling chamber and a second cooling chamber;
the first cooling chamber and the second cooling chamber are in gas communication through a connection conduit; and
a first end of the connection conduit is located inside a portion of the first cooling chamber filled with a gaseous-state cooling medium; and
a second end of the connection conduit is located inside a portion of the second cooling chamber filled with the gaseous-state cooling medium; and
a radiation source configured to emit a radiation beam toward the subject, the radiation source being positioned between the first cooling chamber and the second cooling chamber such that the first cooling chamber and the second cooling chamber are outside of a radiation range of the radiation beam.

32-33. (canceled)
